(19) United States
(12) Patent Application Publication
(10) Pub. No.: US 2023/0322906 A1
(43) Pub. Date: Oct. 12, 2023

(54) HIV-1 SPECIFIC ANTIBODIES AND USES THEREOF

(71) Applicant: VANDERBILT UNIVERSITY, Nashville, TN (US)

(72) Inventors: Lauren Walker, Nashville, TN (US); Ivelin Georgiev, Nashville, TN (US); Andrea R. Shiakolas, Nashville, TN (US); Rohit Venkat, Nashville, TN (US)

(21) Appl. No.: 18/185,067

(22) Filed: Mar. 16, 2023

Related U.S. Application Data
(60) Provisional application No. 63/320,401, filed on Mar. 16, 2022.

(51) Int. Cl. C07K 16/10; A61P 31/18

(52) U.S. Cl. CPC C07K 16/1045; C07K 2317/565; A61P 31/18

(57) ABSTRACT

The present disclosure relates to recombinant antibodies and uses thereof for treating infection.

### CROSS REFERENCE TO RELATED APPLICATIONS

This application claims the benefit of U.S. Provisional Patent Application Ser. No. 63/320,401 filed Mar. 16, 2022, the disclosure of which is expressly incorporated herein by reference in its entirety.

### REFERENCE TO A SEQUENCE LISTING

The Sequence Listing submitted Mar. 16, 2023 as an XML file named “10644-146US1.xml,” created on Mar. 5, 2023, and having a size of 14,499,840 bytes is hereby incorporated by reference pursuant to 37 C.F.R. § 1.831 through 1.835.

### FIELD

The present disclosure relates to antibodies and uses thereof for treating infection.

### BACKGROUND

Due to their high target specificity and relatively low incidence of adverse effects, monoclonal antibody therapeutics have become a mainstay in treatment options for a variety of human diseases such as infection, cancer, autoimmunity, and a multitude of others. For the last several decades antibodies have been used effectively in the clinic to treat patients and one area where considerable effort has gone into developing antibodies for treatment and prevention of infection is HIV-1. Although there has been considerable progress made in drugs used to treat infection such as anti-retrovirals (ART), the lack of a licensed HIV-1 vaccine and the emergence of resistance to current ART regimes emphasize the need for novel treatment options. What is needed are novel compositions and methods for treating HIV-1 infection.

### SUMMARY

Disclosed herein are recombinant antibodies and uses thereof for preventing, treating, and detecting a viral infection (e.g., HIV-1 infection). In some aspects, disclosed herein is a recombinant antibody, wherein the antibody comprises a light chain variable region (VL) that comprises a light chain complementarity determining region (CDRL)1, CDRL2, and CDRL3 and/or a heavy chain variable region (VH) that comprises a heavy chain complementarity determining region (CDRH)1, CDRH2, and CDRH3, wherein: CDRH3 comprises an amino acid sequence at least 60% identical to SEQ ID NOs: 127-144, and wherein CDRL3 comprises an amino acid sequence at least 60% identical to SEQ ID NOs: 145-162.

In some embodiments, the CDRH3 comprises at least one amino acid substitution when compared to SEQ ID NOs: 127-144. In some embodiments, the CDRL3 comprises at least one amino acid substitution when compared to SEQ ID NOs: SEQ ID NOs: 145-162.

In some embodiments, the CDRH1 comprises an amino acid sequence at least 60% identical to SEQ ID NOs: 55-72; and/or the CDRL1 comprises an amino acid sequence at least 60% identical to SEQ ID NOs: 73-90.

In some embodiments, the CDRH2 comprises an amino acid sequence at least 60% identical to SEQ ID NOs: 91-108; and/or the CDRL2 comprises an amino acid sequence at least 60% identical to SEQ ID NOs: 109-126.

In some embodiments, the VH comprises an amino acid sequence selected from the group consisting of SEQ ID NOs: 1-36. In some embodiments, the VL comprises an amino acid sequence selected from the group consisting of SEQ ID NOs: 37-54.

In some embodiments, the CDRH1 comprises the amino acid sequence of SEQ ID NO: 55, the CDRH2 comprises the amino acid sequence of SEQ ID NO: 91, the CDRH3 comprises the amino acid sequence of SEQ ID NO: 127, the CDRL1 comprises the amino acid sequence of SEQ ID NO: 73, the CDRL2 comprises the amino acid sequence of SEQ ID NO: 109, and the CDRL3 comprises the amino acid sequence of SEQ ID NO: 145.

In some embodiments, the CDRH1 comprises the amino acid sequence of SEQ ID NO: 56, the CDRH2 comprises the amino acid sequence of SEQ ID NO: 92, the CDRH3 comprises the amino acid sequence of SEQ ID NO: 128, the CDRL1 comprises the amino acid sequence of SEQ ID NO: 74, the CDRL2 comprises the amino acid sequence of SEQ ID NO: 110, and the CDRL3 comprises the amino acid sequence of SEQ ID NO: 146.

In some embodiments, the CDRH1 comprises the amino acid sequence of SEQ ID NO: 57, the CDRH2 comprises the amino acid sequence of SEQ ID NO: 93, the CDRH3 comprises the amino acid sequence of SEQ ID NO: 129, the CDRL1 comprises the amino acid sequence of SEQ ID NO: 75, the CDRL2 comprises the amino acid sequence of SEQ ID NO: 111, and the CDRL3 comprises the amino acid sequence of SEQ ID NO: 147.

In some embodiments, the CDRH1 comprises the amino acid sequence of SEQ ID NO: 58, the CDRH2 comprises the amino acid sequence of SEQ ID NO: 94, the CDRH3 comprises the amino acid sequence of SEQ ID NO: 130, the CDRL1 comprises the amino acid sequence of SEQ ID NO: 76, the CDRL2 comprises the amino acid sequence of SEQ ID NO: 112, and the CDRL3 comprises the amino acid sequence of SEQ ID NO: 148.

In some embodiments, the CDRH1 comprises the amino acid sequence of SEQ ID NO: 59, the CDRH2 comprises the amino acid sequence of SEQ ID NO: 95, the CDRH3 comprises the amino acid sequence of SEQ ID NO: 131, the CDRL1 comprises the amino acid sequence of SEQ ID NO: 77, the CDRL2 comprises the amino acid sequence of SEQ ID NO: 113, and the CDRL3 comprises the amino acid sequence of SEQ ID NO: 149.

In some embodiments, the CDRH1 comprises the amino acid sequence of SEQ ID NO: 60, the CDRH2 comprises the amino acid sequence of SEQ ID NO: 96, the CDRH3 comprises the amino acid sequence of SEQ ID NO: 132, the CDRL1 comprises the amino acid sequence of SEQ ID NO: 78, the CDRL2 comprises the amino acid sequence of SEQ ID NO: 114, and the CDRL3 comprises the amino acid sequence of SEQ ID NO: 150.

In some embodiments, the CDRH1 comprises the amino acid sequence of SEQ ID NO: 61, the CDRH2 comprises the amino acid sequence of SEQ ID NO: 97, the CDRH3 comprises the amino acid sequence of SEQ ID NO: 133, the CDRL1 comprises the amino acid sequence of SEQ ID NO: 79, the CDRL2 comprises the amino acid sequence of SEQ ID NO: 115, and the CDRL3 comprises the amino acid sequence of SEQ ID NO: 151.

In some embodiments, the CDRH1 comprises the amino acid sequence of SEQ ID NO: 62, the CDRH2 comprises the amino acid sequence of SEQ ID NO: 98, the CDRH3 comprises the amino acid sequence of SEQ ID NO: 134, the CDRL1 comprises the amino acid sequence of SEQ ID NO: 80, the CDRL2 comprises the amino acid sequence of SEQ ID NO: 116, and the CDRL3 comprises the amino acid sequence of SEQ ID NO: 152.

In some embodiments, the CDRH1 comprises the amino acid sequence of SEQ ID NO: 63, the CDRH2 comprises the amino acid sequence of SEQ ID NO: 99, the CDRH3 comprises the amino acid sequence of SEQ ID NO: 135, the CDRL1 comprises the amino acid sequence of SEQ ID NO: 81, the CDRL2 comprises the amino acid sequence of SEQ ID NO: 117, and the CDRL3 comprises the amino acid sequence of SEQ ID NO: 153.

In some embodiments, the CDRH1 comprises the amino acid sequence of SEQ ID NO: 64, the CDRH2 comprises the amino acid sequence of SEQ ID NO: 100, the CDRH3 comprises the amino acid sequence of SEQ ID NO: 136, the CDRL1 comprises the amino acid sequence of SEQ ID NO: 82, the CDRL2 comprises the amino acid sequence of SEQ ID NO: 118, and the CDRL3 comprises the amino acid sequence of SEQ ID NO: 154.

In some embodiments, the CDRH1 comprises the amino acid sequence of SEQ ID NO: 65, the CDRH2 comprises the amino acid sequence of SEQ ID NO: 101, the CDRH3 comprises the amino acid sequence of SEQ ID NO: 137, the CDRL1 comprises the amino acid sequence of SEQ

ID NO: 83, the CDRL2 comprises the amino acid sequence of SEQ ID NO: 119, and the CDRL3 comprises the amino acid sequence of SEQ ID NO: 155.

In some embodiments, the CDRH1 comprises the amino acid sequence of SEQ ID NO: 66, the CDRH2 comprises the amino acid sequence of SEQ ID NO: 102, the CDRH3 comprises the amino acid sequence of SEQ ID NO: 138, the CDRL1 comprises the amino acid sequence of SEQ ID NO: 84, the CDRL2 comprises the amino acid sequence of SEQ ID NO: 120, and the CDRL3 comprises the amino acid sequence of SEQ ID NO: 156.

In some embodiments, the CDRH1 comprises the amino acid sequence of SEQ ID NO: 67, the CDRH2 comprises the amino acid sequence of SEQ ID NO: 103, the CDRH3 comprises the amino acid sequence of SEQ ID NO: 139, the CDRL1 comprises the amino acid sequence of SEQ ID NO: 85, the CDRL2 comprises the amino acid sequence of SEQ ID NO: 121, and the CDRL3 comprises the amino acid sequence of SEQ ID NO: 157.

In some embodiments, the CDRH1 comprises the amino acid sequence of SEQ ID NO: 68, the CDRH2 comprises the amino acid sequence of SEQ ID NO: 104, the CDRH3 comprises the amino acid sequence of SEQ ID NO: 140, the CDRL1 comprises the amino acid sequence of SEQ ID NO: 86, the CDRL2 comprises the amino acid sequence of SEQ ID NO: 122, and the CDRL3 comprises the amino acid sequence of SEQ ID NO: 158.

In some embodiments, the CDRH1 comprises the amino acid sequence of SEQ ID NO: 69, the CDRH2 comprises the amino acid sequence of SEQ ID NO: 105, the CDRH3 comprises the amino acid sequence of SEQ ID NO: 141, the CDRL1 comprises the amino acid sequence of SEQ ID NO: 87, the CDRL2 comprises the amino acid sequence of SEQ ID NO: 123, and the CDRL3 comprises the amino acid sequence of SEQ ID NO: 159.

In some embodiments, the CDRH1 comprises the amino acid sequence of SEQ ID NO: 70, the CDRH2 comprises the amino acid sequence of SEQ ID NO: 106, the CDRH3 comprises the amino acid sequence of SEQ ID NO: 142, the CDRL1 comprises the amino acid sequence of SEQ ID NO: 88, the CDRL2 comprises the amino acid sequence of SEQ ID NO: 124, and the CDRL3 comprises the amino acid sequence of SEQ ID NO: 160.

In some embodiments, the CDRH1 comprises the amino acid sequence of SEQ ID NO: 71, the CDRH2 comprises the amino acid sequence of SEQ ID NO: 107, the CDRH3 comprises the amino acid sequence of SEQ ID NO: 143, the CDRL1 comprises the amino acid sequence of SEQ ID NO: 89, the CDRL2 comprises the amino acid sequence of SEQ ID NO: 125, and the CDRL3 comprises the amino acid sequence of SEQ ID NO: 161.

In some embodiments, the CDRH1 comprises the amino acid sequence of SEQ ID NO: 72, the CDRH2 comprises the amino acid sequence of SEQ ID NO: 108, the CDRH3 comprises the amino acid sequence of SEQ ID NO: 144, the CDRL1 comprises the amino acid sequence of SEQ ID NO: 90, the CDRL2 comprises the amino acid sequence of SEQ ID NO: 126, and the CDRL3 comprises the amino acid sequence of SEQ ID NO: 162.

In some aspects, disclosed herein is a method of treating, preventing, and/or mitigating a viral infection in a subject in need comprising administering to the subject a therapeutically effective amount of a recombinant antibody of any preceding aspect.

### BRIEF DESCRIPTION OF DRAWINGS

The accompanying figures, which are incorporated in and constitute a part of this

specification, illustrate aspects described below.

FIGS. 1A-1C show identification of CD4bs and V3-glycan site specific antibodies from HIV-1 infected subjects. (FIG. 1A) Schematic of DNA barcoded antigens used in LIBRA-seq with epitope mapping assays. (FIG. 1B) Cells (dots) from experiments 1 (left, donors N16 and N76) and 2 (right, N55) are shown. For a given cell, the minimum LIBRA-seq score among all antigen variants for a given epitope (CD4bs, V3-glycan, V2) was computed, and the lowest (x-axis) and second-lowest (y-axis) epitope scores are plotted. Each dot is colored by the LIBRA-seq score for BG505sc.SOSIP (backbone), from lowest (blue) to highest (red). Antibodies selected for validation are shown in black outline. (FIG. 1C) Sequence characteristics and LIBRA-seq score information for candidate antibodies. Percent identity was calculated at the nucleotide level, and CDR3 length and sequences are displayed at the amino acid level. LIBRA-seq scores for each antigen are displayed on a scale of light yellow(low)-white-purple(high). The sequences in FIGS. 1A-1C are CARGGSLWFGREFHAMDVW (SEQ ID NO: 163), CARGGQLWFGERLYFHFW (SEQ ID NO: 164), CARGGQLWFGERLNFQHW (SEQ ID NO: 165), CARDIVTIFFRGRSQHAFSVW (SEQ ID NO: 166), CARERITVFFRGRSQHASNVW (SEQ ID NO: 167), CARKVWPFNGMDVW (SEQ ID NO: 168), CASEPVKIFGVVSLGEVFYVGMDVW (SEQ ID NO: 169), CLLSYSGARRVF (SEQ ID NO: 170), CQQSFSTPITF (SEQ ID NO: 171), CQQSHSTPLTF (SEQ ID NO: 172), CQVWDISSDHQVF (SEQ ID NO: 173), CQVWDSSSDHQVF (SEQ ID NO: 174), CQQLTNYATF (SEQ ID NO: 175), QSTDMNSIYKT (SEQ ID NO: 176).

FIGS. 2A-2C show validation and characterization of CD4bs and V3-glycan specific antibodies discovered through LIBRA-seq. (FIG. 2A) A heat map of ELISA AUC values for binding to BG505.SOSIP variant proteins are shown for each antibody. AUC values for each variant were normalized to BG505.SOSIP.backbone for each antibody. ELISA binding curves located in the supplement. (FIG. 2B) HIV-1 Env/CD4 inhibition ELISA is shown for all antibodies. CD4bs antibody VRC01 was used as a positive control and outer domain glycan specific antibody 2G12 was used as a negative control. Data is shown as % CD4 blocking by each antibody. (FIG. 2C) Competition ELISA of 4591-1 with antibodies PGT121, 3074, 2G12, and 10-1074. Antibody VRC01 was used as a negative control. Data is shown as % inhibition of binding by the reference antibody. White indicates <25% inhibition, grey indicates 25-75% inhibition and black represents >75% inhibition. All ELISAs were performed in technical duplicate with 2 biological duplicates; data represented as mean±SEM.

FIGS. 3A-3C show identification of confirmational specific antibodies from LIBRA-seq with epitope mapping. (FIG. 3A) Schematic of DNA barcoded antigens used in LIBRA-seq with epitope mapping assays. (FIG. 3B) Upset plot for all B cells with LIBRA-seq score >1 against different HIV-1 antigen combinations. On the X-axis, each column represents a different combination of antigens (rows), showing which antigens are part of a given combination (displayed by filled circles), with the bar height corresponding to the number of B cells identified to be reactive with the given antigen combination. Each combination is mutually exclusive. The total number of B cells predictive to be reactive with each individual antigen is indicated as a horizontal bar at the bottom left of each subpanel. Isotype for each antibody is depicted by different colors. (FIG. 3C) LIBRA-seq scores and ELISA AUC values for each antibody binding to HIV-1 trimer and monomer antigens. Scores are represented by heatmaps, with the lowest scores displayed as light yellow and the highest as purple. ELISA data are representative of at least two different experiments. LIBRA-seq score data shown for H9 Hong Kong 2009 HA and ELISA AUC data shown for A/New Caledonia/20/99 H1N1 (NC99) HA. ELISA binding curves located in supplement.

FIG. 4 shows identification of HIV-1 SOSIP Specific B Cells using LIBRA-seq. LIBRA-seq score information for all cells from N76/N16 LIBRA-seq run and N55 LIBRA-seq run. LIBRA-seq score information for each antigen is displayed on a scale from light yellow(low)-white-purple (high).

FIGS. 5A-5B show validation and characterization of CD4bs and V3-glycan specific antibodies discovered through LIBRA-seq. (FIG. 5A) ELISA binding curves for binding to BG505.SOSIP variant proteins are shown for antibodies: 6420-35, 6420-48.1, 6420-48.2, 6420-233.1, 6420-233.2, 4591-2 and 4591-1. Flu HA protein was used as a negative control antigen. (FIG. 5B) ELISA binding curves for binding to YU2 gp120 core and YU2 gp120 core D368R proteins are shown for antibodies: 6420-35, 6420-48.1, 6420-48.2, 6420-233.1, 6420-233.2, and 4591-2. Absorbance values at 450nm shown on Y-axis. Antibody concentration in μg/ml shown on the X-axis.

FIG. 6 shows identification of HIV-1 monomer and trimer specific B cells using LIBRA-seq. LIBRA-seq score information for all cells from N27 LIBRA-seq run and N26 LIBRA-seq run. LIBRA-seq score information for each antigen is displayed on a scale from light yellow(low)-white-purple(high).

FIGS. 7A-7B show validation and characterization of monomer and trimer specific antibodies discovered through LIBRA-seq. (FIG. 7A) Sequence characteristics for candidate antibodies. Percent identity was calculated at the nucleotide level, and CDR length and sequences are displayed at the amino acid level. (FIG. 7B) ELISA binding curves for binding to HIV-1 trimeric and monomeric proteins are shown for candidate antibodies. Flu HA protein used as a negative control antigen. Absorbance values at 450nm shown on Y-axis. Antibody concentration in μg/ml shown on the X-axis. The sequences in FIGS. 7A-7B include CARDPAAGRSYYYYYVDVW (SEQ ID NO: 177), CARMFYESSTGYPSWFDPW (SEQ ID NO: 178), CAYRLEHPSNWYDYFHHW (SEQ ID NO: 179), CATSSGVVSLRSAFDIW (SEQ ID NO: 180), CARDGLRLNFYYYGMDVW (SEQ ID NO: 181), CARVGQPKFFERSSDGEHFQHW (SEQ ID NO: 182), CARDVRMRSGRPRQYCMDVW (SEQ ID NO: 183), CAKDWSFDDDGGAYVPSAMDVW (SEQ ID NO: 184), CARDFYYDSSGYYPDAFDIW (SEQ ID NO: 185), CARDIGLGRGHDYW (SEQ ID NO: 186), CARGRHSVVEPAAKYRFDTW (SEQ ID NO: 187), CQSYDSNLSGVVF (SEQ ID NO: 188), CQQYFYTPWTF (SEQ ID NO: 189), CQQYNFHSPTF (SEQ ID NO: 190), CCSYVGSNTWVF (SEQ ID NO: 191), CQQNGESPRMF (SEQ ID NO: 192), CHQYSRYPWTF (SEQ ID NO: 193), CQQYDTSPAVTF (SEQ ID NO: 194), CQQYGSSPFTF (SEQ ID NO: 195), CQQYNNGRTF (SEQ ID NO: 196), CSSYIAYNILEVF (SEQ ID NO: 197), and CQQSYSTPPTF (SEQ ID NO: 198).

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same 30 meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “antigen” refers to a molecule that is capable of binding to an antibody. In some embodiments, the antigen stimulates an immune response such as by production of antibodies specific for the antigen.

In the present invention, “specific for” and “specificity” means a condition where one of the molecules is involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. Native antibodies are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In human, there are 51 VH, 27 DH, 6 RI, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called α, δ, ϵ, γ, and μ, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv, nanoantibody and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M.J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The terms “antigen binding site”, “binding site” and “binding domain” refer to the specific elements, parts or amino acid residues of a polypeptide, such as an antibody, that bind the antigenic determinant or epitope.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

The term “CDR” as used herein refers to the “complementarity determining regions” of the antibody which consist of the antigen binding loops. (Kabat E.A. et al., (1991) Sequences of proteins of immunological interest. NIH Publication 91-3242). Each of the two variable domains of an antibody Fv fragment contain, for example, three CDRs.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. The amino acid sequence boundaries of a CDR can be determined by one of skill in the art using any of a number of known numbering schemes, including those described by Kabat et al., supra (“Kabat” numbering scheme): Al-Lazikani et al., 1997. J. Mol. Biol., 273:927-948 (“Chothia” numbering scheme); MacCallum et al., 1996, J. Mol. Biol, 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a bacterium, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to an antigen, and/or ameliorating an infection.

The term “identity” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.).

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells trans-formed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “specificity” refers to the number of different types of antigens or antigenic determinants to which a particular antigen-binding molecule (such as the recombinant antibody of the invention) can bind. As used herein, the term “specifically binds,” as used herein with respect to a recombinant antibody refers to the recombinant antibody's preferential binding to one or more epitopes as compared with other epitopes. Specific binding can depend upon binding affinity and the stringency of the conditions under which the binding is conducted. In one example, an antibody specifically binds an epitope when there is high affinity binding under stringent conditions.

It should be understood that the specificity of an antigen-binding molecule (e.g., the recombinant antibodies of the present invention) can be determined based on affinity and/or avidity. The affinity, represented by the equilibrium constant for the dissociation of an antigen with an antigen-binding molecule (K_{D}), is a measure for the binding strength between an antigenic determinant and an antigen-binding site on the antigen-binding molecule: the lesser the value of the KD, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as the recombinant antibodies of the present invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the recombinant antibodies of the invention) will bind to their antigen with a dissociation constant (K_{D}) of 10⁻⁵ to 10⁻¹² moles/liter or less, and preferably 10⁻⁷ to 10⁻¹² moles/liter or less, and more preferably 10⁻⁸ to 10⁻¹² moles/liter.

“Therapeutically effective amount” refers to the amount of a composition such as recombinant antibody that will elicit the biological or medical response of a tissue, system, animal, or human that is being sought by the researcher, veterinarian, medical doctor or other clinician over a generalized period of time. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount of recombinant antibodies as described herein can be determined by one of ordinary skill in the art.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, such as decreased viral/bacterial titers, decreased viral RNA levels, and/or prolonged survival of a subject. It will be understood that the total daily usage of the compositions and formulations as disclosed herein will be decided by the attending physician within the scope of sound medical judgment. The exact amount required will vary depending on factors such as the type of disease being treated.

The terms “treat,” “treating,” “treatment,” and grammatical variations thereof as used herein, include partially or completely delaying, alleviating, mitigating or reducing the intensity of one or more attendant symptoms of a condition or infection and/or alleviating, mitigating or impeding one or more causes of a condition or infection. Treatments according to the invention may be applied preventively, prophylactically, palliatively or remedially. Prophylactic treatments are administered to a subject prior to onset (e.g., before obvious signs of an infection), during early onset (e.g., upon initial signs and symptoms of an infection), after an established development of an infection, or during chronic infection. Prophylactic administration can occur for several minutes to months prior to the manifestation of an infection.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

### Antibodies and Methods

In some aspects, disclosed herein is a recombinant antibody, wherein the antibody comprises a light chain variable region (VL) comprising a light chain complementarity determining region (CDRL)1, CDRL2, and CDRL3 and/or a heavy chain variable region (VH) that comprises a heavy chain complementarity determining region (CDRH)1, CDRH2, and CDRH3, wherein: CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 127-144; and CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 145-162.

In some embodiments, the CDRH3 comprises at least one (e.g., at least one, two, three, four, five, six, seven, eight, nine, or ten) amino acid substitution when compared to SEQ ID NOs: 127-144. In some embodiments, the CDRL3 comprises at least one (e.g., at least one, two, three, four, five, six, seven, eight, nine, or ten) amino acid substitution when compared to SEQ ID NOs: SEQ ID NOs: 145-162.

In some embodiments, the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 55-72; and/or the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 73-90.

In some embodiments, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 91-108; and/or the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 109-126.

In some embodiments, disclosed herein is a recombinant antibody, wherein the VH comprises an amino acid sequence selected from the group consisting of SEQ ID NOs: 1-36. In some embodiments, the VL comprises an amino acid sequence selected from the group consisting of SEQ ID NOs: 37-54.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 55, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 91, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 127, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 73, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 109, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 145.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 56, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 92, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 128, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 74, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 110, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 146.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 57, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 93, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 129, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 75, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 111, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 147.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 58, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 94, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 130, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 76, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 112, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 148.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 59, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 95, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 131, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 77, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 113, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 149.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 60, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 96, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 132, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 78, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 114, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 150.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 61, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 97, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 133, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 79, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 115, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 151.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 62, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 98, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 134, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 80, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 116, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 152.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 63, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 99, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 135, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 81, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 117, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 153.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 64, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 100, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 136, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 82, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 118, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 154.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 65, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 101, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 137, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 83, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 119, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 155.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 66, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 102, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 138, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 84, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 120, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 156.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 67, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 103, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 139, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 85, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 121, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 157.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 68, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 104, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 140, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 86, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 122, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 158.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 69, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 105, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 141, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 87, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 123, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 159.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 70, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 106, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 142, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 88, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 124, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 160.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 71, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 107, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 143, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 89, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 125, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 161.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 72, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 108, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 144, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 90, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 126, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 162.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 19 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 37.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 20 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 38.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 21 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 39.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 22 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 40.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 23 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 41.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 24 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 42.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 25 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 43.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 26 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 44.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 27 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 45.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 28 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 46.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 29 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 47.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 30 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 48.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 31 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 49.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 32 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 50.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 33 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 51.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 34 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 52.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 35 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 53.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 36 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 54.

In some aspects, disclosed herein is a method for treating, reducing, decreasing, inhibiting, and/or preventing an infectious disease in a subject in need thereof comprising administering to the subject a therapeutically effective amount of any of the antibodies disclosed herein. In some embodiments, the infectious disease is caused by infection of a virus, such as, for example, an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-0C43, HCoV-HKU1, HCoV-NL63, SARS-CoV, SARS-CoV-2, or MERS-CoV), Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In some aspects, disclosed herein is a method for treating, reducing, decreasing, inhibiting, and/or preventing a viral infection in a subject in need thereof comprising administering to the subject a therapeutically effective amount of the antibodies disclosed herein.

In some embodiments, the method comprises administering to the subject a therapeutically effective amount of a recombinant antibody, wherein the antibody comprises a light chain variable region (VL) that comprises a light chain complementarity determining region (CDRL)1, CDRL2, and CDRL3 and/or a heavy chain variable region (VH) that comprises a heavy chain complementarity determining region (CDRH)1, CDRH2, and CDRH3, wherein: CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 127-144; and CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 145-162.

In some embodiments, the CDRH3 comprises at least one amino acid substitution when compared to SEQ ID NOs: 127-144. In some embodiments, the CDRL3 comprises at least one amino acid substitution when compared to SEQ ID NOs: SEQ ID NOs: 145-162.

In some embodiments, the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 55-72; and/or the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 73-90.

In some embodiments, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 91-108; and/or the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 109-126.

In some embodiments, disclosed herein is a recombinant antibody, wherein the VH comprises an amino acid sequence selected from the group consisting of SEQ ID NOs: 1-36. In some embodiments, the VL comprises an amino acid sequence selected from the group consisting of SEQ ID NOs: 37-54.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 55, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 91, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 127, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 73, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 109, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 145.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 56, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 92, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 128, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 74, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 110, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 146.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 57, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 93, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 129, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 75, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 111, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 147.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 58, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 94, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 130, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 76, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 112, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 148.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 59, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 95, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 131, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 77, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 113, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 149.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 60, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 96, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 132, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 78, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 114, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 150.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 61, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 97, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 133, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 79, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 115, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 151.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 62, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 98, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 134, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 80, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 116, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 152.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 63, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 99, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 135, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 81, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 117, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 153.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 64, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 100, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 136, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 82, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 118, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 154.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 65, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 101, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 137, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 83, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 119, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 155.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 66, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 102, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 138, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 84, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 120, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 156.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 67, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 103, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 139, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 85, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 121, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 157.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 68, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 104, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 140, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 86, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 122, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 158.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 69, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 105, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 141, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 87, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 123, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 159.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 70, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 106, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 142, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 88, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 124, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 160.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 71, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 107, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 143, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 89, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 125, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 161.

In some aspects, disclosed herein is a recombinant antibody, wherein the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 72, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 108, the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 144, the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 90, the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 126, and the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 162.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 19 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 37.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 20 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 38.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 21 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 39.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 22 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 40.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 23 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 41.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 24 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 42.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 25 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 43.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 26 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 44.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 27 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 45.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 28 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 46.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID

NO: 29 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 47.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 30 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 48.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 31 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 49.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 32 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 50.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 33 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 51.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 34 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 52.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 35 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 53.

In some embodiments, the VH comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 36 and the VL comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 54.

In some embodiments, the antibody described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

As the timing of an infection or onset of a related disease can often not be predicted, it should be understood the disclosed methods of treating, inhibiting, reducing, ameliorating, and/or preventing the disease or disorder described herein can be used prior to or following the onset of the infection or the disease, to treat, prevent, inhibit, and/or reduce the infection or the disease. In one aspect, the disclosed methods can be employed 30, 29, 28, 27, 26, 25, 24, 23, 22, 21, 20, 19, 30 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of the infection or the disease; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the infection or the disease.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

### EXAMPLES

The following examples are set forth below to illustrate the antibodies, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

### High-Throughput B Cell Epitope Determination by Next-Generation Sequencing

Antibodies represent an attractive therapeutic option in this context, and as such, multiple antibodies are currently being investigated in clinical trials. All antibodies being tested in the clinic target the HIV-1 envelope glycoprotein (Env), the sole target for neutralizing antibodies. There are multiple antigenic regions on Env that have been shown to be important epitopes targeted by neutralizing antibody responses, including the CD4-binding site (CD4bs), glycan-dependent epitopes (V1V2 and V3), membrane-proximal external region (MPER), gp120-gp41 interface, and others.

Technologies to discover and develop new antibody therapeutics such as hybridoma generation, antigen-specific B cell sorting, phage or yeast display, and single-cell B cell sequencing, have made the process generally quicker and more efficient. An underlying goal of antibody discovery technologies is to identify paired heavy and light chain B cell receptor (BCR) sequences from antigen-specific B cells. This is then followed up by subsequent antibody production and characterization. However, only limited information about antigen and epitope specificity is obtained during initial B cell or antibody screening steps, often requiring the profiling of tens to hundreds of antibodies to identify the few with epitopes or functions of interest. In order to overcome these obstacles, LIBRA-seq was developed, a next-generation single-cell sequencing technology for simultaneous recovery of BCR sequence and antigen specificity by utilizing DNA-barcoded antigens. LIBRA-seq has successfully been applied to identify antibodies against infectious agents such as HIV-1, influenza, and a number of coronaviruses.

This example aimed to increase the power and utility of LIBRA-seq to include simultaneous recovery of B cell receptor sequence and paired antigen epitope specificity at the level of individual epitope residues. To this end, this study developed a panel of DNA-barcoded HIV-1 Env epitope-specific variants, to distinguish epitope-specific B cells, and allowing for residue-level epitope binding information to be transformed into a sequence-able event. LIBRA-seq was applied with epitope mapping to samples from chronically-infected HIV-1 donors, leading to the discovery of antibodies specific to multiple different Env epitope regions, including the CD4bs and V3-glycan sites. In addition, this study utilized an antigen panel of pre-fusion stabilized Env gp140 trimers along with monomeric gp120 proteins to identify conformation-dependent B cells, allowing domain-level epitope binding information to be transformed into a sequence-able event. LIBRA-seq with epitope mapping is a high-throughput antibody discovery platform that enables the identification of residue-level epitope information through next-generation sequencing, therefore reducing the burden of epitope-specific antibody discovery away from the costly and laborious subsequent steps of antibody production and characterization.

### Materials and Methods

Human Subjects. HIV-1 infected donors were enrolled in investigational review board approved clinical protocols at the National Institutes of Allergy and Infectious Diseases (NIAID). Peripheral blood mononuclear cells (PBMCs) were collected from donors NIAID26 (N26), NIAID27 (N27), NIAID55 (N55), NIAID76 (N76) and NIAID16 (N16). Collection dates were as follows: 9/30/2009, 6/16/2006, 11/6/2008, 12/12/2007 and 7/2/2007. All donors were chronically infected at the time of sample collection. It is likely all patients were infected with clade B strain of HIV-1.

Antigen Production. In total, four LIBRA-seq experiments were performed. In one experiment, seven HIV-1 gp140 BG505.SOSIP variants were used: single-chain BG505sc.SOSIP.T332N (backbone sequence for all BG505.SOSIP variants), BG505sc. SOSIP.N332T, BG505sc.SOSIP.N279K, BG505sc. SOSIP.D368R/N279K, BG505sc.SOSIP.N160K, BG505sc.SOSIP.K169E, BG505sc.SOSIP.D368R, and one Influenza hemagglutinin variant A/New Caledonia/20/99 H1N1 (NC99). These antigens were used to identify antigen-positive B cells from peripheral blood mononuclear cells (PBMCs) from donors N16 and N76. In experiment 2, the antigen panel from experiment 1 was used, with the following modifications: BG505.6R.SOSIP.T332N replaced BG505sc.SOSIP.T332N, seven HIV-1 gp140 CZA97. SOSIP variants were added (CZA97sc. SOSIP.N332T, CZA97sc.SOSIP.N160K, CZA97sc.SOSIP.K169E, CZA97sc. SOSIP.N279K, CZA97sc. SOSIP.D368R/N279K, CZA97sc.SOSIP.D368R, CZA97.6R.SOSIP.T332N (backbone sequence for all CZA97.SOSIP variants), and hepatitis C antigen JFH-1 E2c was also added. These antigens were used to sort antigen positive B cells from PBMCs from donor N55. In the final two LIBRA-seq experiments, HIV-1 gp140 SOSIP antigens CZA97, ZM197 and CNE55 were utilized along with HIV-1 gp120 proteins, A244 and ConC, and influenza hemagglutinin variants, A/Michigan/45/2015 H1N1, A/Indonesia/5/2005 H5N1, A/Anhui/1/2013 H7N9, H9 Hong Kong 2009 HA, H10 Jiangxi-Donghu 2013 HA. These antigens were used to sort antigen positive B cells from donors N26 and N27.

Recombinant HIV-1 trimer and HCV proteins were expressed in Expi 293F mammalian cells (Thermo Fisher) with PEI transfection reagent and cultured for 5-7 days in FreeStyle F17 expression Medium supplemented (Thermo Fisher) with 10% Pluronic acid and 20% glutamine. Cells were maintained at 37° C. with 8% CO2 saturation while shaking. After 5-7 days cells were spun down and supernatant was harvested. Supernatant was run over an affinity column of agarose-bound Galanthus nivalis lectin (GNA, Snowdrop). The column was washed with 1X PBS and bound protein was eluted with 1M methyl-a-D-mannopyranoside. Protein elute was then buffer exchanged into 1X PBS and further purified by size exclusion chromatography with a Superdex 200 Increase 10/300 GL Sizing column on the AKTA FPLC system (GE Life Sciences). Purified protein was analyzed by SDS-PAGE and characterized by ELISA.

Recombinant ConC gp120 and A244 gp120 with an Avi-tag at the C-terminus were expressed in HEK 293 F cells using PEI-max (Polysciences) for 72 h. Culture supernatant was harvested and centrifuged to remove cells and cell debris. The cleared supernatant was filtered through 0.22 stericup filters (Millipore) and the soluble gp120 proteins were then purified by affinity chromatography by passing the supernatant through a column of agarose bound Glanathus nivalus lectin (Sigma). The column was then washed with 1× PBS and proteins were eluted with 1 M methyl α-D-mannopyranoside (Sigma). The eluted proteins were concentrated using a 30 KDa cut-off vivaspin filters and further purified by ion exchange chromatography on a on a HiPrep™ Q HP 16/10 anion exchange column. The gp120 fraction was then collected and buffer exchanged and the proteins concentrated using a 30 Kda cut-off vivaspin filter. The purified proteins were tested for antigenicity by ELISA and the purity was checked by SDS-PAGE.

Recombinant HA proteins (A/New Caledonia/20/99 H1N1 GenBank ACF41878 (NC99), A/Michigan/45/2015 H1N1 GenBank AMA11475, A/Indonesia/5/2005 H5N1 GenBank ABP51969, and A/Anhui/1/2013 H7N9 GISAID EPI439507, A/Hong Kong/33982/2009 H9N2 GISAID EPI470900, A/Jaingxi-Donghu/246/2013 H1ON8 GISAID EPI497477) all contained the HA ectodomain with a point mutation at the sialic acid-binding site (Y98F), T4 fibritin foldon trimerization domain, AviTag, and hexahistidine-tag, and were expressed in Expi 293F mammalian cells using Expifectamine 293 transfection reagent (Thermo Fisher Scientific) cultured for 4-5 days. Culture supernatant was harvested and cleared as above, and then adjusted pH and NaCl concentration by adding 1M Tris-HCl (pH 7.5) and 5 M NaCl to 50 mM and 500 mM, respectively. Ni Sepharose excel resin (GE Healthcare) was added to the supernatant to capture hexahistidine tag. Resin was separated on a column by gravity and captured HA protein was eluted by a Tris-NaCl (pH 7.5) buffer containing 300 mM imidazole. The elute was further purified by a size exclusion chromatography with a HiLoad 16/60 Superdex 200 column (GE Healthcare). Fractions containing HA were concentrated, analyzed by SDS-PAGE and tested for antigenicity by ELISA with known antibodies. Proteins were frozen at −80 C until use.

AviTagged antigens were biotinylated using BirA biotin ligase (Avidity LLC).

DNA-Barcoding of Antigens. This study used oligos that possess 15 bp antigen barcode, a sequence capable of annealing to the template switch oligo that is part of the 10× bead-delivered oligos and contain truncated TruSeq small RNA read 1 sequences in the following structure: 5′-CCTTGGCACCCGAGAATTCCA CCCATATAAGA*A*A-3′ (SEQ ID NO: 232), where Ns represent the antigen barcode. For each antigen, a unique DNA barcode was directly conjugated to the antigen itself. In particular, 5′amino-oligonucleotides were conjugated directly to each antigen using the Solulink Protein-Oligonucleotide Conjugation Kit (TriLink cat no. S-9011) according to manufacturer's instructions. Briefly, the oligo and protein were desalted, and then the amino-oligo was modified with the 4FB crosslinker, and the biotinylated antigen protein was modified with S-HyNic. Then, the 4FB-oligo and the HyNic-antigen were mixed together. This causes a stable bond to form between the protein and the oligonucleotide. The concentration of the antigen-oligo conjugates was determined by a BCA assay, and the HyNic molar substitution ratio of the antigen-oligo conjugates was analyzed using the NanoDrop according to the Solulink protocol guidelines. AKTA FPLC was used to remove excess oligonucleotide from the protein-oligo conjugates, which were also verified using SDS-PAGE with a silver stain. Antigen-oligo conjugates were also used in flow cytometry titration experiments. The following barcodes were used for the donors N16 and N76 experiments: ATTCGCCTTACGCAA (SEQ ID NO: 199) (BG505sc.SOSIP.T332N), AACCCACCGTTGTTA (SEQ ID NO: 200) (BG505sc.SOSIP.N332T), GGTAGCCCTAGAGTA (SEQ ID NO: 201) (BG505sc.SOSIP.N279K), CAGTAAGTTCGGGAC (SEQ ID NO: 202) (BG505sc.SOSIP.DKO), CTTCACTCTGTCAGG (SEQ ID NO: 203) (BG505sc.SOSIP.N160K), TACGCCTATAACTTG (SEQ ID NO: 204) (BG505sc.SOSIP.K169E), AGACTAATAGCTGAC (SEQ ID NO: 205) (BG505sc.SOSIP.D368R), and GCTCCTTTACACGTA (SEQ ID NO: 206) (A/New Caledonia/20/99 H1N1 (NC99)). The following barcodes were used for the donor N55 experiment: GCAGCGTATAAGTCA (SEQ ID NO: 207) (BG505sc.SOSIP.T332N), GCTCCTTTACACGTA (SEQ ID NO: 208) (BG505sc.SOSIP.N332T), CTTCACTCTGTCAGG (SEQ ID NO: 209) (BG505sc.SOSIP.N279K), TGGTAACGACAGTCC (SEQ ID NO: 210) (BG505sc.SOSIP.DKO), TGTGTATTCCCTTGT (SEQ ID NO: 211) (BG505sc.SOSIP.N160K), TACGCCTATAACTTG (SEQ ID NO: 212) (BG505sc.SOSIP.K169E), GTGTGTTGTCCTATG (SEQ ID NO: 213) (BG505sc.SOSIP.D368R), AACCCACCGTTGTTA (SEQ ID NO: 214) (A/New Caledonia/20/99 H1N1 (NC99)); CAGTAAGTTCGGGAC (SEQ ID NO: 215), GTAAGACGCCTATGC (SEQ ID NO: 216), TTTCAACGCCCTTTC (SEQ ID NO: 217), CCGTCCTGATAGATG (SEQ ID NO: 218), TCATTTCCTCCGATT (SEQ ID NO: 219), GGTAGCCCTAGAGTA (SEQ ID NO: 220), AGACTAATAGCTGAC (SEQ ID NO: 221) (CZA97 antigens). The following barcodes were used for the donors N26 and N27 experiments: CAGCCCACTGCAATA (SEQ ID NO: 222) (CZA97), ATCGTCGAGAGCTAG (SEQ ID NO: 223) (ZM197), TCACAGTTCCTTGGA (SEQ ID NO: 224) (CNE55), CAGATGATCCACCAT (SEQ ID NO: 225) (A244), GACCTCATTGTGAAT (SEQ ID NO: 226) (ConC), TGACCTTCCTCTCCT (SEQ ID NO: 227) (A/Michigan/45/2015 H1N1), CAGGTCCCTTATTTC (SEQ ID NO: 228) (A/Indonesia/5/2005 H5N1), ACAATTTGTCTGCGA (SEQ ID NO: 229) (A/Anhui/1/2013 H7N9), AACCTTCCGTCTAAG (SEQ ID NO: 230) (H9 Hong Kong 2009 HA), AATCACGGTCCTTGT (SEQ ID NO: 231) (H10 Jiangxi-Donghu 2013 HA).

Antigen-Specific B Cell Sorting. For each sample, PBMCs were mixed with DNA-barcoded fluorescently labeled antigens, stained with fluorescent cell markers and single cell sorted using fluorescence-activated cell sorting (FACS). Briefly, cells were thawed and washed twice with DPBS 0.1% BSA. For donors NIAID16 and NIAID76 cells were stained with antibodies against cell markers including viability dye (Ghost Red 780), CD14-APC-Cy7, IgM-APC-Cy7 CD3-FITC, CD19-BV711, and IgG-PE-Cy5. For donor NIAID55 cells were stained with antibodies against cell markers including (CD3-APCCy7, IgG-FITC, CD19-BV711, CD14-V500). For donors NIAID26 and NIAID27 cells were stained with antibodies against cell markers including viability dye (Ghost Red 780), CD14-APC-Cy7, CD3-FITC, CD19-BV711, and IgG-PE-Cy5. The cell-antigen mixture was incubated in the dark for 15-30 minutes. The cells were then washed twice with DPBS 0.1% BSA and stained with Streptavidin-PE (1:1000) for 15-30 minutes in the dark. The cells were washed again twice with DPBS 0.1% BSA and taken to the Flow Cytometry core for single cell sorting by FACS. Antigen positive cells were collected and delivered to the sequencing core VANTAGE for single-cell processing and sequencing.

Sample Preparation, Library Preparation, and Sequencing. Single-cell suspensions were be loaded onto the Chrommium microfluidics device (10× Genomics) and processed using the B-cell VDJ solution according to manufacturer's suggestions. Minor modifications were made in order to separate the antigen and cellular mRNA barcode libraries.

Sequence processing and bioinformatics analysis. Paired-end FASTQ files of oligo libraries were used as input and processed using a previously described pipeline. Reads for cell barcode, UMI and antigen barcodes were used to generate a cell barcode-antigen barcode UMI count matrix. BCR contigs were processed using Cell Ranger (10× Genomics) using GRCh38 as reference. Antigen barcode libraries were also processed using Cell Ranger (10× Genomics). The overlapping cell barcodes between the two libraries were used as the basis of the subsequent analysis. Cell barcodes that had only non-functional heavy chain sequences as well as cells with multiple functional heavy chain sequences and/or multiple functional light chain sequences were removed. Additionally, the BCR contigs (filtered_contigs.fasta file output by Cell Ranger, 10× Genomics) was aligned to IMGT reference genes using HighV-Quest20. The output of HighV-Quest was parsed using ChangeO21 and merged with an antigen barcode UMI count matrix. In experiments utilizing the panel of DNA-barcoded HIV-1 Env epitope-specific variants, the LIBRA-seq score was determined. In experiments utilizing stabilized Env trimers ZM197, CNE55, CZA97, along with monomeric gp120 proteins from strains A244 and ConC, the LIBRA-seq score was determined. Next, to prioritize BCR sequences for recombinant antibody production and characterization, further filtering steps were applied; cells with UMI scores for negative control antigens >20 and cells for which the max antigen UMI <10 were not considered for further analysis. Due to consistently poor UMI signal for the CZA97.SOSIP backbone across cells, the data for CZA97. SOSIP variants for donor N55 was not included in further analysis.

Antibody Expression and Purification. For each antibody, variable gene sequences were inserted into custom plasmids encoding the heavy chain IgG1 constant region and the corresponding lamda or kappa light chain region (pTwist 314 CMV BetaGlobin WPRE Neo vector, Twist Bioscience). Antibodies were expressed in Expi293F mammalian cells (Thermo Fisher) with PEI transfection reagent and cultured for 5-7 days in FreeStyle F17 expression Medium supplemented with 10% Pluronic acid and 20% glutamine. Cells were maintained at 37° C. with 8% CO₂ saturation while shaking. After 5-7 days cells were spun down and supernatant was harvested. Supernatant was run over a protein A affinity column. The column was washed with 1×PBS and the protein was eluted with 100 mM Glycine HCl at 2.7 pH directly into a 1:10 volume of 1M Tris-HCl pH 8.0. Eluted antibodies were buffer exchanged using Amicon Ultra-centrifugal filter units into 1X PBS and stored for future use.

ELISA. To assess antibody binding, soluble protein was plated on Immulon 2HB plates at 2 μg/mL overnight at 4° C. In cases where capture ELISA was used, plates were pre-incubated overnight at 4° C. with 2 μg/ml anti-AviTag (GenScript) and washed 3× with PBS+0.05% Tween-20 (PBS-T) before antigen plating for 2 hr at room temperature (RT). Plates were washed three times with PBS supplemented with 0.05% Tween-20 (PBS-T) and coated with 5% milk powder in PBS-T. Plates were incubated for one hour at RT. Plates were washed three times with PBS-T. Primary antibodies were diluted in 1% milk in PBS-T, starting at 10 μg/m with a serial 1:10 dilution and then added to the plate. The plates were incubated at RT for one hour and then washed three times in PBS-T. The secondary antibody, goat anti-human IgG conjugated to peroxidase (Thermo Fisher), was added at 1:10,000 dilution in 1% milk in PBS-T to the plates, which were incubated for one hour at RT. Plates were washed three times with PBS-T and then developed by adding TMB substrate (Thermo Fisher) to each well. The plates were incubated at room temperature for ten minutes, and then 1N sulfuric acid was added to stop the reaction. Plates were read at 450 nm. Data are represented as either mean±SEM for one ELISA experiment or %AUC normalized to BG505.SOSIP.backbone. ELISAs were repeated 2 or more times. The area under the curve (AUC) was calculated using Prism software version 8.0.0 (GraphPad).

CD4 Binding Inhibition Assay. 96-well plates were coated with 2 μg/mL purified recombinant BG505sc.backbone at 4° C. overnight. The next day, plates were washed three times with PBS-T and coated with 5% milk powder in PBS-T. Plates were incubated for one hour at RT and then washed three times with PBS-T. Purified antibodies were diluted in blocking buffer at 50 μg/mL in duplicate, added to the wells, and incubated at RT. Without washing, biotinylated recombinant human CD4-Fc tag protein (Sino Biological) was added to wells for a final 20 μg/mL concentration of CD4-Fc and incubated for 30 minutes at RT. Plates were washed three times with PBS-T, and bound CD4-Fc was detected using Streptavidin-HRP (Thermo Fisher) and TMB substrate. The plates were incubated at room temperature for ten minutes, and then 1N sulfuric acid was added to stop the reaction. Plates were read at 450 nm. CD4-Fc binding without antibody served as a control. Experiments were done in biological replicate and technical duplicate.

Competition ELISA. Competition ELISA experiments were performed as above with minor modifications. After coating with antigen and blocking, non-biotinylated competitor antibody was added to each well at 20 μg/ml and incubated at RT for 30 mins. After washing, biotinylated antibody (final concentration of 2 μg/ml) was added and incubated for 30 mins at RT. After washing three times with PBS-T, streptavidin-HRP was added at 1:10,000 dilution in 1% milk powder in PBS-T and incubated for 1 hour at room temperature. Plates were washed and substrate and sulfuric acid were added as described above.

Results

Targeted Identification of CD4 Binding Site and V3 Glycan Site Directed Antibodies Using LIBRA-seq

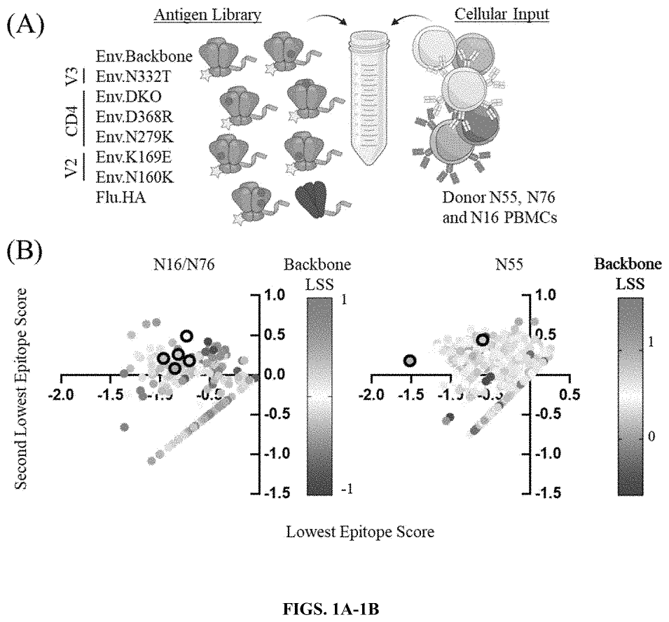

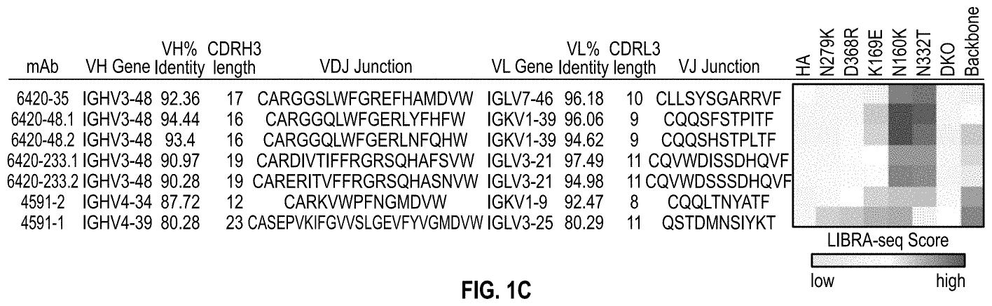

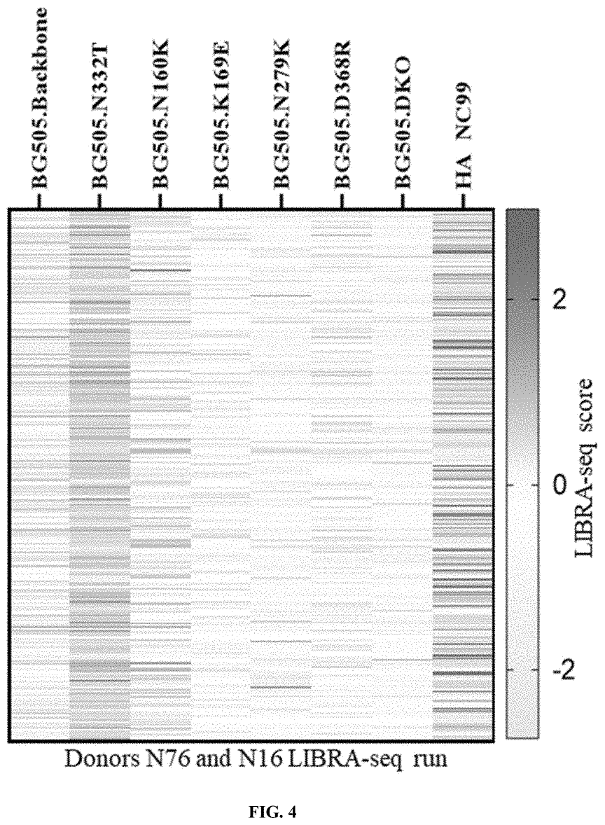

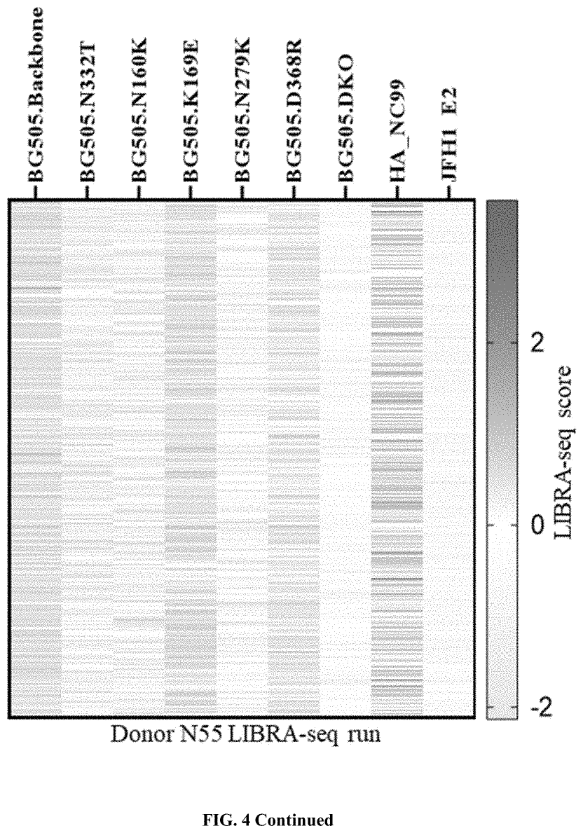

To demonstrate the feasibility of the LIBRA-seq with epitope mapping technology, HIV-1 Env epitope-specific antibodies were identified from chronically infected HIV-1 donors. In two separate experiments, a screen was performed for antigen-positive B cells using a panel of pre-fusion stabilized, soluble Env variants that contained point mutations in distinct antigenic regions on the HIV-1 Env protein (Clade A/BG505). Each antigen was labeled with a unique DNA barcode and a fluorescent tag for fluorescence-activated cell sorting (FACS). In the screening libraries, included were a backbone Env strain (BG505.SOSIP.T332N), three CD4bs mutants (BG505.SOSIP.D368R, BG505.SOSIP.N279K, and BG505.SOSIP.D368R/N279K (referred to as double knockout (DKO)); two V2 loop mutants (BG505.SOSIP.K169E, and BG505.SOSIP.N160K), and one V3-glycan site mutant (BG505.SOSIP.N332T) (FIG. 1A). Together, these two experiments resulted in the identification of 2,442 B cells with antigen specificity and BCR sequence information (FIG. 4).

Next, the recombinant expression of a set of B cell receptorswas prioritized based on the criteria that the cells displayed a distinct epitope signature across the different Env variants, such as a low LIBRA-seq score for a given epitope and high LIBRA-seq scores for the respective backbone and other epitope variants. Among the candidate B cells, several cells were selected for validation with CD4bs epitope specificity signal by LIBRA-seq (FIGS. 1B and 1C). Specifically, two lineages were selected with two clonally related antibody members in each lineage, and two additional antibodies that did not belong to either clonal lineage. These antibodies, 6420-35, 6420-48.1, 6420-48.2, 6420-233.1, 6420-233.2 and 4591-2, displayed low LIBRA-seq scores for the BG505.SOSIP CD4bs variants and high LIBRA-seq scores for the backbone, V2 and V3-glycan BG505.SOSIP variants (FIGS. 1B and 1C). Five of the antibodies utilized the IGHV3-48 heavy chain variable gene and one antibody utilized the IGHV4-34 heavy chain variable gene. The antibodies displayed moderate to high levels of heavy chain somatic hypermutation (6-12%) and CDRH3 lengths of 12-19 amino acids (FIG. 1C). The antibodies utilized several different light chain germline genes (IGLV7-46, IGKV1-39, IGLV3-21 and IGKV1-9). In addition to the CD4bs antibodies, an additional antibody was selected for validation with a predicted V3-glycan epitope specificity. This antibody, 4591-1, displayed a low LIBRA-seq score for the BG505.SOSIP V3-glycan variant N332T and high LIBRA-seq scores for the BG505.SOSIP.backbone, V2 and CD4bs variants (FIG. 1B and 1C). This antibody utilized variable heavy chain gene IGHV4-39 and displayed a high level of somatic hypermutation (20%) and a CDRH3 length of 22 amino acids (FIG. 1C).

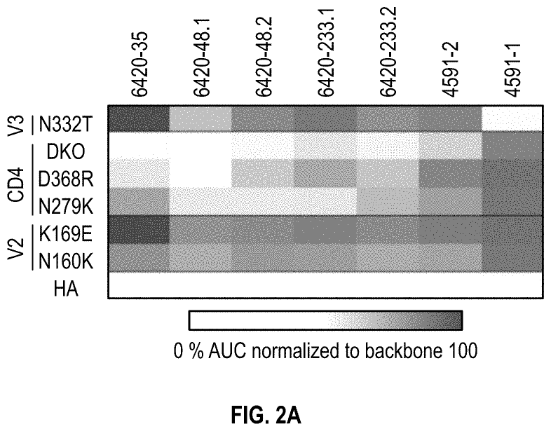

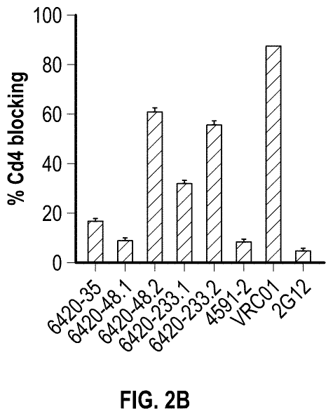

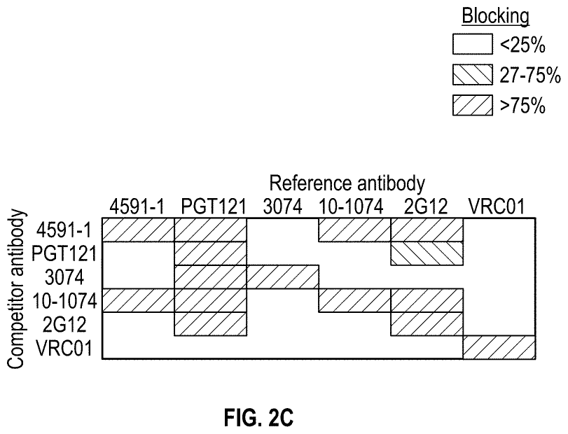

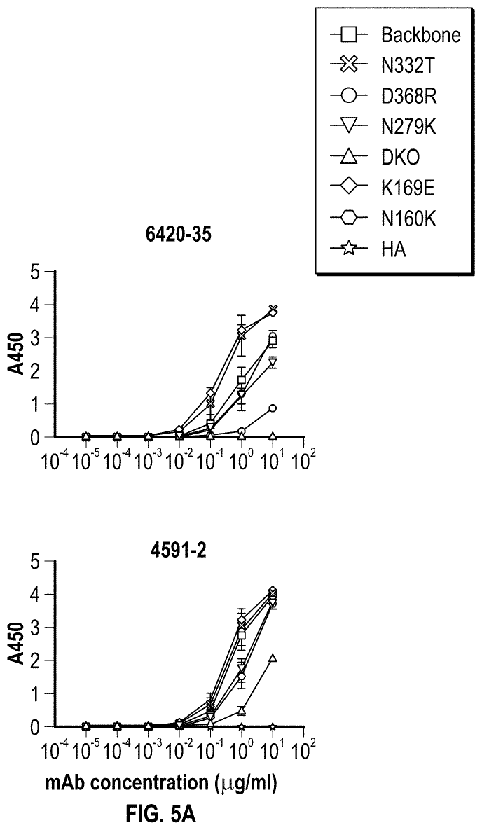

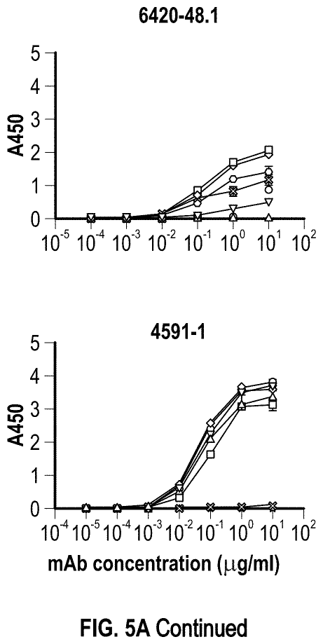

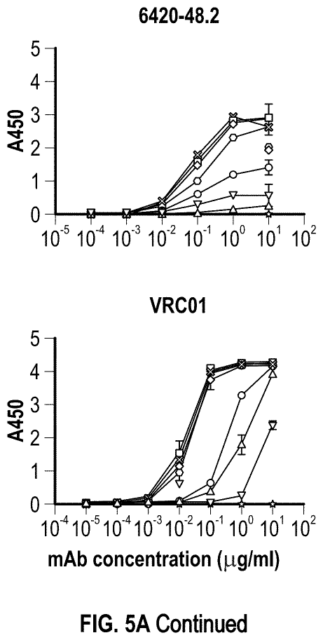

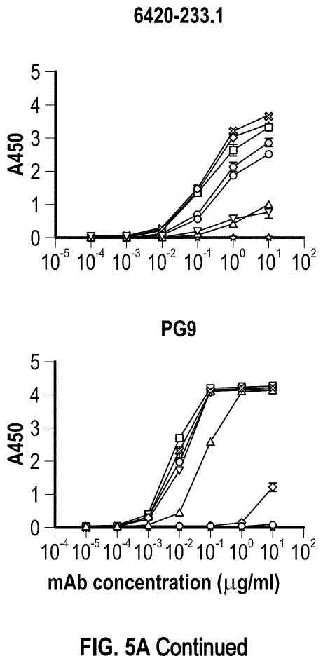

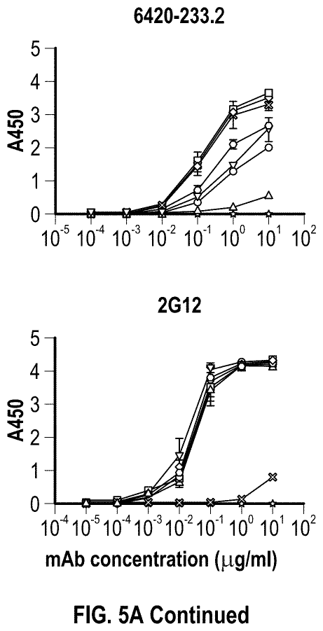

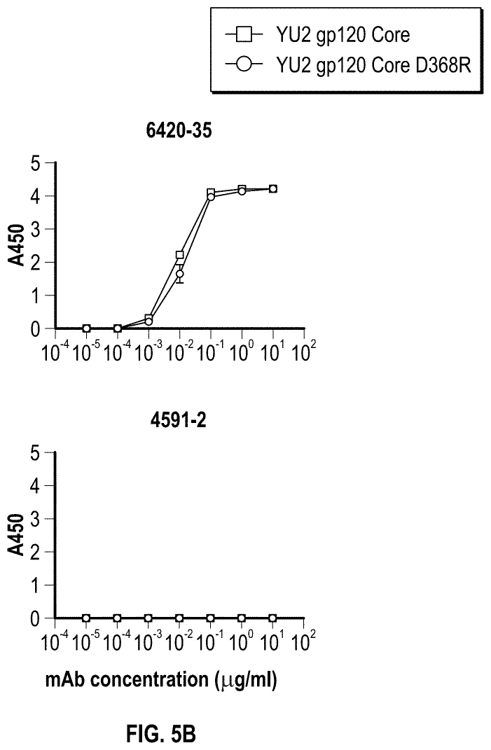

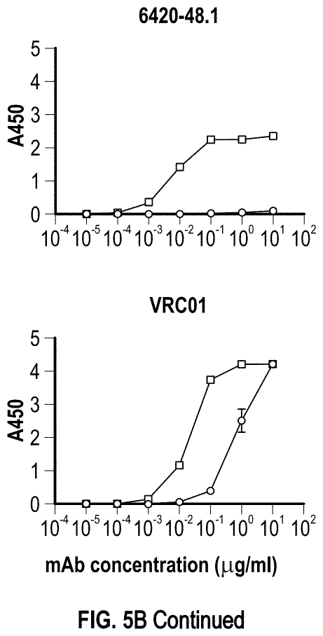

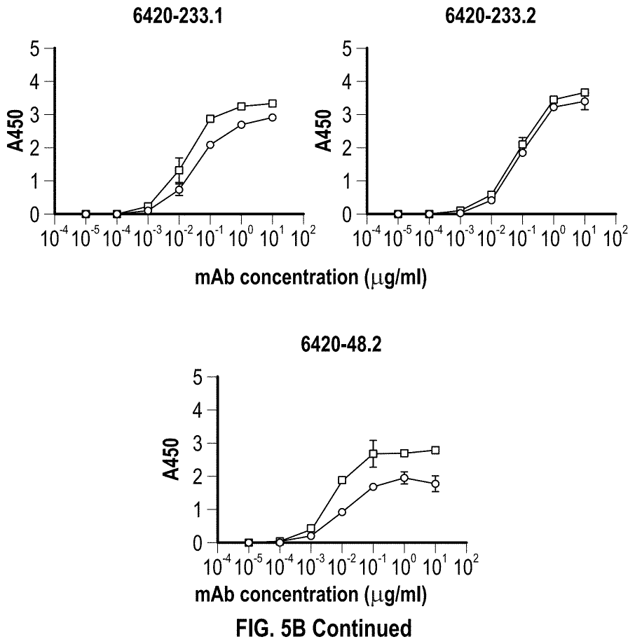

To confirm the epitope specificity predicted by LIBRA-seq for the selected antibodies, tests were performed for binding by ELISA to BG505. SOSIP. Notably, all six antibodies exhibited binding to this antigen, confirming the HIV-1 Env specificity of all of these antibodies (FIG. 5A). Next, these antibodies were tested against the different epitope-specific BG505.SOSIP Env variants (FIG. 2A). The binding of antibodies 6420-35, 6420-48.1, 6420-48.2, 6420-233.1, 6420-233.2 and 4591-2 to BG505.SOSIP Env was associated with a >2-fold decrease in binding for one or more of the CD4bs variants, but not for the V2 or V3-glycan variants (FIG. 2A and FIG. 5A). Further, the antibodies were tested for their ability to bind YU2 gp120 core protein, which lacks variable regions V1, V2 and V3, and its respective CD4bs mutant D368R. Binding to YU2 gp120 D368R was affected to a different extent for the six antibodies, suggesting that sensitivity to this specific CD4bs mutation may be context-specific (FIG. 5B). Finally, the ability of these six antibodies to block the interaction between HIV-1 Env and its cognate receptor CD4 was tested, using a soluble, Fc-tagged CD4. The antibodies showed different levels of CD4-blocking ability by ELISA (FIG. 2B). Together, these results suggest that these six antibodies target the CD4bs epitope on HIV-1 Env, as predicted by LIBRA-seq.

Next experiment investigated the epitope specificity of antibody 4591-1, and found that antibody binding to Env was inhibited by the BG505.SOSIP V3-glycan mutation N332T, but not by mutations in the V2 or CD4bs regions, as predicted by LIBRA-seq (FIG. 2A and FIG. 5A). To further confirm the 4591-1 epitope, the experiment tested the ability of this antibody to compete for binding to BG505.SOSIP.backbone Env with known V3-glycan antibodies PGT121, 3074, 2G12 and 10-1074. It was found that 4591-1 competed with antibodies PGT121, 2G12 and 10-1074, but did not compete with V3-crown specific antibody 3074 or CD4bs antibody VRC01 (FIG. 2C). Together, these results indicate that antibody 4591-1 targets the V3-glycan epitope on HIV-1 Env, as predicted by LIBRA-seq.

Overall, these data indicate that the LIBRA-seq technology can be successfully applied to identify antibodies against specific epitopes of interest on a target antigen.

Targeted Identification of Monomer- and Trimer-Specific Antibodies Using LIBRA-seq

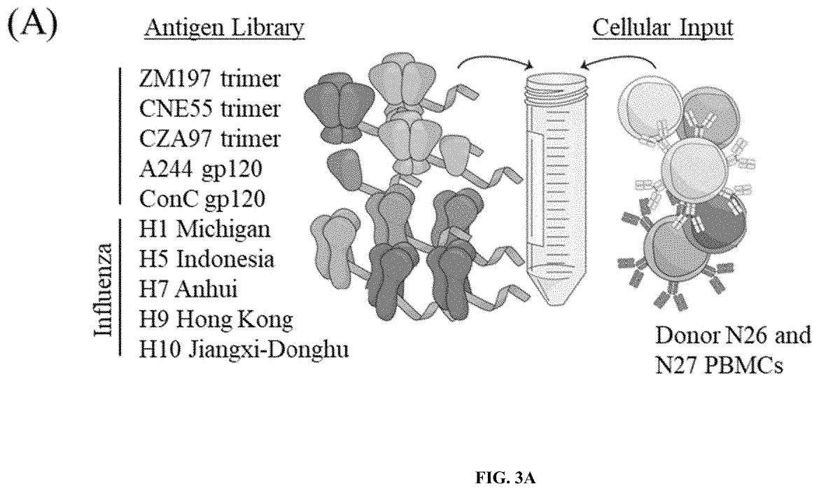

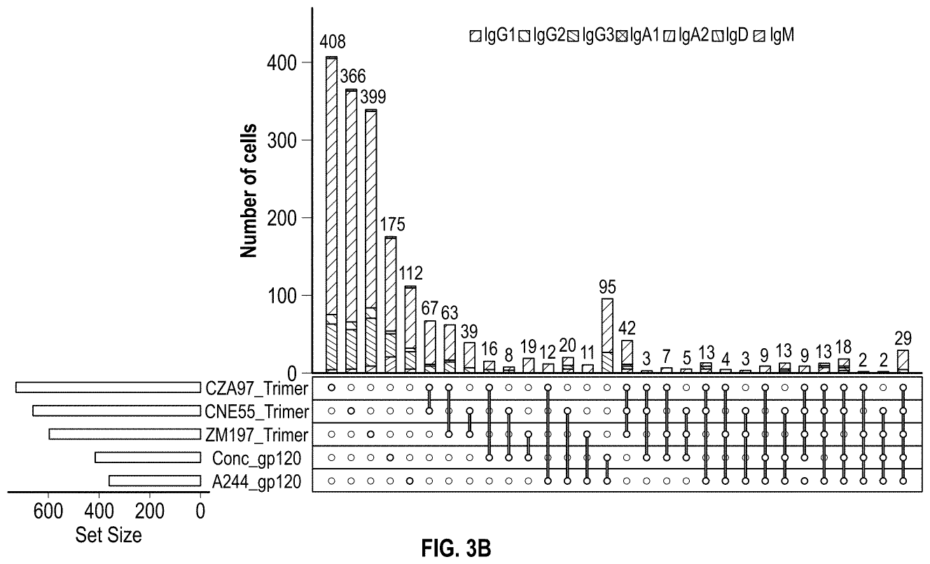

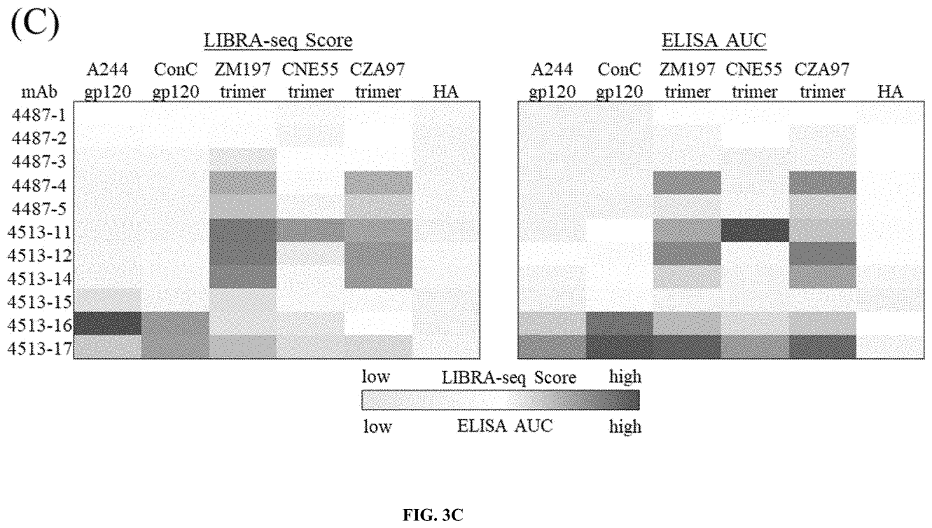

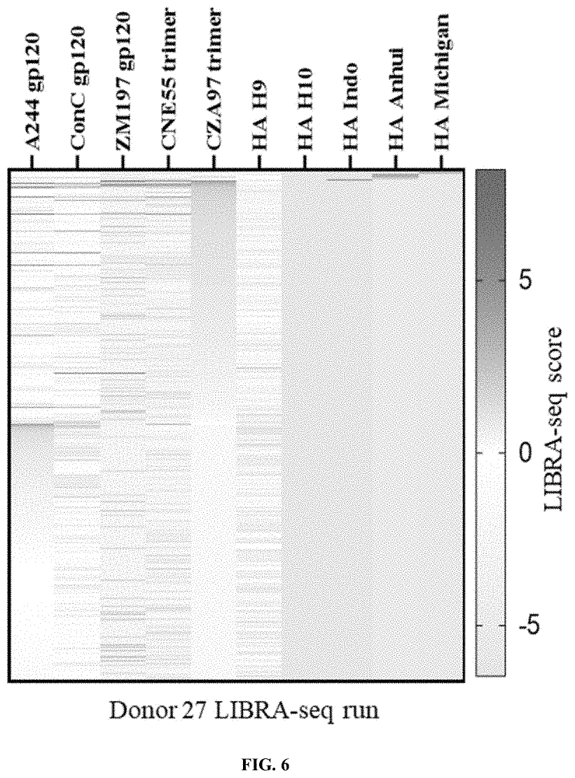

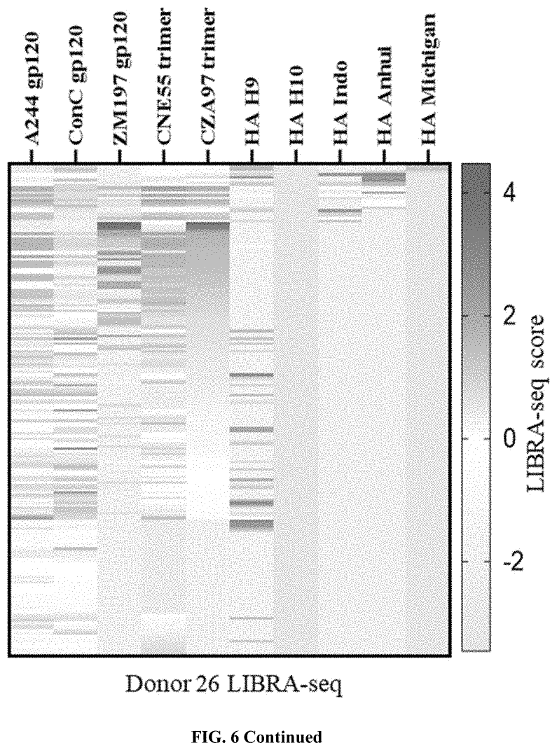

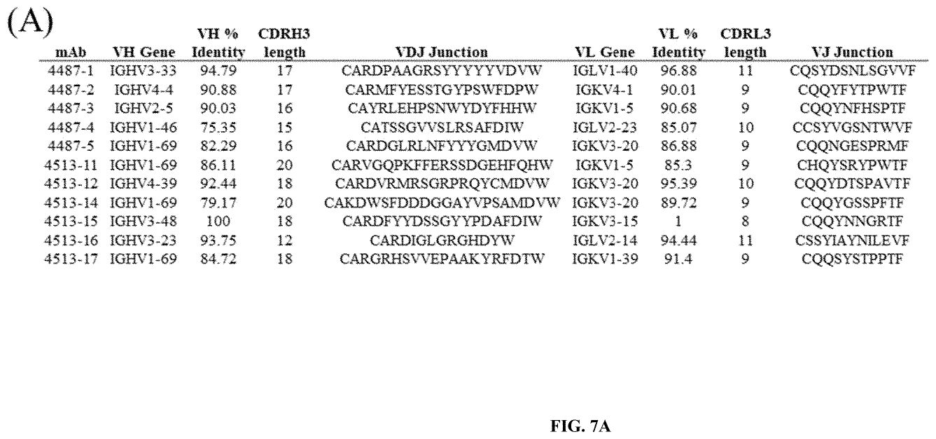

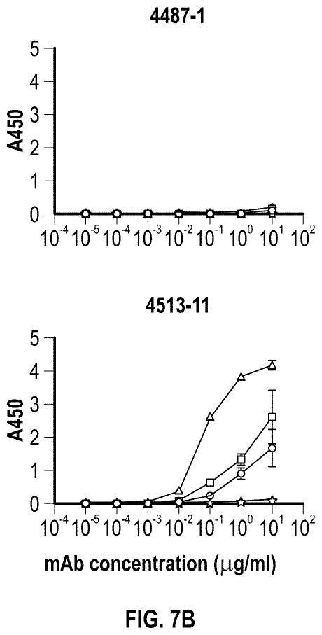

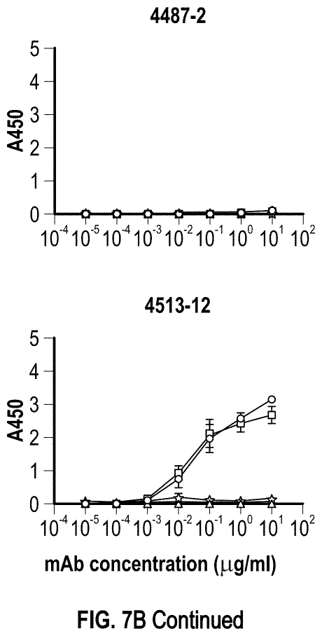

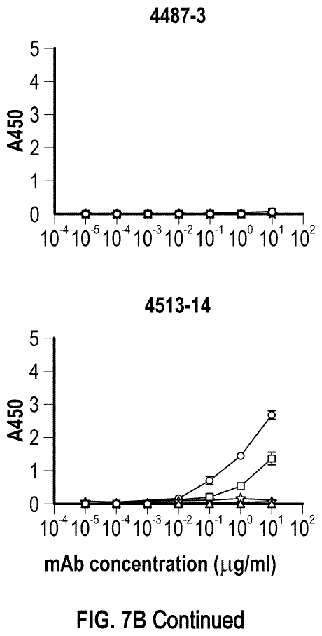

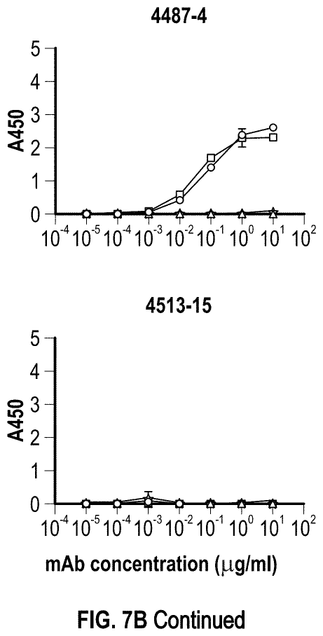

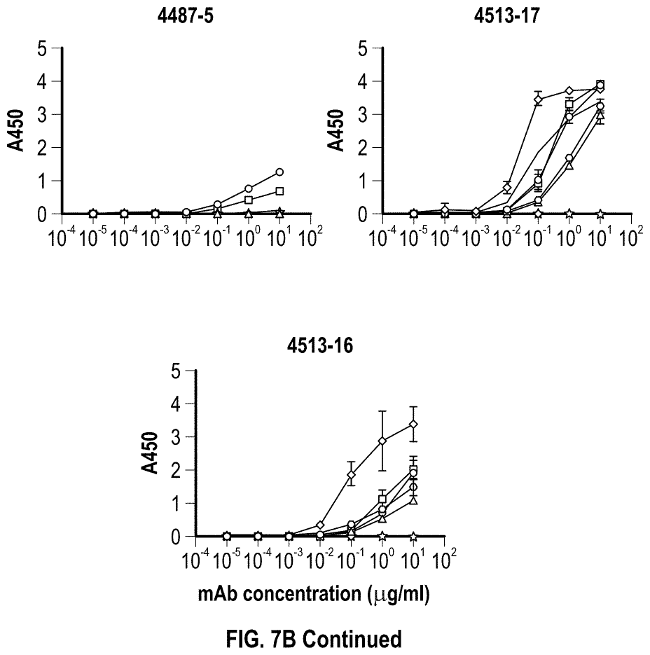

In addition to the ability of LIBRA-seq to screen antibody candidates for residue-level epitope specificity, this study sought to apply this technology toward identifying antibody candidates that specifically recognize different conformational states of the target antigen. HIV-1 Env represents an appropriate target for such an experiment since antigen variants in different conformational states are available. In order to determine the ability of LIBRA-seq to identify trimeric vs monomeric anti-Env antibodies, this experiment utilized an antigen panel of pre-fusion stabilized Env gp140 trimers from multiple clade C strains (ZM197, CNE55, CZA97), along with monomeric gp120 proteins from clades AE and C (AE/A244 and C/ConC). (FIG. 3A). These two experiments together resulted in the identification of 3,843 cells with available antigen specificity and BCR sequence information (FIG. 6). In particular, patterns of trimer-only reactivity, monomer-only reactivity, or both monomer and trimer reactivity for a variety of B cells were observed (FIG. 6). Two sets of B cells were then prioritized for recombinant monoclonal antibody production and characterization. Set one (antibodies 4513-15, 4513-16 and 4513-17) displayed positive LIBRA-seq scores for at least one trimer and at least one monomer (FIG. 3C). Set two (antibodies 4513-11, 4513-14, 4513-12, 4487-1, 4487-2, 4487-3. 4487-4, and 4487-5) displayed positive LIBRA-seq scores for at least one trimer and low LIBRA-seq scores for the two monomers (FIG. 3C). Interestingly, four out of the eleven antibodies, 4513-17, 4513-14, 4513-11 and 4487-5 utilized the variable heavy chain gene IGHV1-69, which has been shown to be commonly utilized during viral infections including HIV-1, although all appeared to be from different B cell lineages (FIG. 7A). Overall, the eleven antibodies exhibited diverse sequence features, including the utilization of multiple heavy and light chain germline genes, a range of CDRH3 lengths, and diverse somatic mutation levels for both the heavy and light chains (FIG. 7A).

To confirm the conformational specificity predicted by LIBRA-seq for the selected antibodies, this study tested them for binding by ELISA to the same panel of HIV-1 Env gp140 trimer and gp120 monomer antigens as used in the LIBRA-seq library (FIG. 3C). Binding to at least one of the antigens was observed for seven of the 11 antibodies, confirming the HIV-1 Env specificity for these antibodies, as predicted by LIBRA-seq (FIG. 3C). As suggested by the LIBRA-seq scores, antibodies 4513-16 and 4513-17 displayed binding to both monomer and trimer antigens (FIG. 3C and Supplementary FIG. 4B). In contrast and also as suggested by the LIBRA-seq scores, antibodies 4513-11, 4513-12, 4513-14, 4487-4 and 4487-5 displayed binding to trimeric Env protein >3 times that to monomeric Env protein, indicating these antibodies preferentially recognize Env in its trimeric conformation state (FIG. 3C and FIG. 7B). Together, these results confirm that LIBRA-seq with epitope mapping can be applied to successfully identify conformation-specific antibodies.

This study describes the application of LIBRA-seq with epitope mapping to identify novel monoclonal antibodies that target diverse sites of vulnerability on the HIV-1 Env glycoprotein. Through high-throughput sequencing experiments, this study discovered and validated the specificity for several antibodies targeting the CD4bs of Env, an antibody targeting the V3-glycan site of Env, and conformation-dependent antibodies that recognize trimeric Env protein over monomeric forms of the protein. While standard epitope mapping technologies can shed light onto the epitopes targeted by antigen-specific antibodies, this is typically done either through bulk serum analysis, or for a small set of individual antibodies. In contrast, by including antigen variants with epitope-knockout mutations in the screening library, LIBRA-seq enables high-throughput, high-dimensional, single-cell epitope analysis. Further, with the generation of the paired heavy-light chain sequence information for each given antibody, LIBRA-seq analysis can also be applied to explore associations between antibody sequence and epitope specificity.

Advances in antibody sequencing technologies have contributed to the increase in therapeutic antibodies available to treat patients. LIBRA-seq with epitope mapping provides critical advantages in the antibody discovery pipeline through high-resolution data generation earlier in the process. The results presented here allow inclusion of epitope specificity as part of the sequencing readout. Prioritizing antibody candidates based on predicted epitope specificity information obtained during the initial sequencing stage can significantly decrease the burden posed by the subsequent production and validation of hundreds to thousands of leads for epitope-specific antibody discovery. More broadly, LIBRA-seq with epitope mapping can be applied to other disease settings where the discovery of epitope specific antibodies is the main priority or to the evaluation of vaccine candidates where understanding what types of antibodies are elicited is critical.

TABLE 1
Antibody Sequences

| | cell barcode | | | | | | | |
|---|---|---|---|---|---|---|---|---|
| | and heavy | | SEQ ID | V-J- | | | | |
| | (HC) or | SEQ ID | NO of | REGION | CDR1- | CDR2- | CDR3- | |
| | light | NO of | V-D-J- | (LC), | IMGT, | IMGT, | IMGT, | Antibodies |
| Sequence | chain (LC) | cell | REGION | SEQ ID | SEQ ID | SEQ ID | SEQ ID | of special |
| number | designation | barcode | (HC) | NO | NO | NO | NO | interest |
| 11855 | ACGTCAAAG | SEQ ID | SEQ ID | | SEQ ID | SEQ ID | SEQ ID | 6420-48.2 |
| | TACGCGA.HC | NO: 1 | NO: 19 | | NO: 55 | NO: 91 | NO: 127 | |
| 11856 | ACGTCAAAG | SEQ ID | | SEQ ID | SEQ ID | SEQ ID | SEQ ID | 6420-48.2 |
| | TACGCGA.LC | NO: 1 | | NO: 37 | NO: 73 | NO: 109 | NO: 145 | |
| 12133 | CTACACCTC | SEQ ID | SEQ ID | | SEQ ID | SEQ ID | SEQ ID | 6420-48.1 |
| | CAAATGC.HC | NO: 2 | NO: 20 | | NO: 56 | NO: 92 | NO: 128 | |
| 12134 | CTACACCTC | SEQ ID | | SEQ ID | SEQ ID | SEQ ID | SEQ ID | 6420-48.1 |
| | CAAATGC.LC | NO: 2 | | NO: 38 | NO: 74 | NO: 110 | NO: 146 | |
| 11885 | AGCATACTC | SEQ ID | SEQ ID | | SEQ ID | SEQ ID | SEQ ID | 6420-35 |
| | ACGGTTA.HC | NO: 3 | NO: 21 | | NO: 57 | NO: 93 | NO: 129 | |
| 11886 | AGCATACTC | SEQ ID | | SEQ ID | SEQ ID | SEQ ID | SEQ ID | 6420-35 |
| | ACGGTTA.LC | NO: 3 | | NO: 39 | NO: 75 | NO: 111 | NO: 147 | |
| 11931 | ATCTACTGT | SEQ ID | SEQ ID | | SEQ ID | SEQ ID | SEQ ID | 6420-233.2 |
| | CGGCACT.HC | NO: 4 | NO: 22 | | NO: 58 | NO: 94 | NO: 130 | |
| 11932 | ATCTACTGT | SEQ ID | | SEQ ID | SEQ ID | SEQ ID | SEQ ID | 6420-233.2 |
| | CGGCACT.LC | NO: 4 | | NO: 40 | NO: 76 | NO: 112 | NO: 148 | |
| 12411 | TAAACCGGT | SEQ ID | SEQ ID | | SEQ ID | SEQ ID | SEQ ID | 6420-233.1 |
| | TATCACG.HC | NO: 5 | NO: 23 | | NO: 59 | NO: 95 | NO: 131 | |
| 12412 | TAAACCGGT | SEQ ID | | SEQ ID | SEQ ID | SEQ ID | SEQ ID | 6420-233.1 |
| | TATCACG.LC | NO: 5 | | NO: 41 | NO: 77 | NO: 113 | NO: 149 | |
| 1405 | CCGTACTAG | SEQ ID | SEQ ID | | SEQ ID | SEQ ID | SEQ ID | 4591-2 |
| | CGAAGGG.HC | NO: 6 | NO: 24 | | NO: 60 | NO: 96 | NO: 132 | |
| 1406 | CCGTACTAG | SEQ ID | | SEQ ID | SEQ ID | SEQ ID | SEQ ID | 4591-2 |
| | CGAAGGG.LC | NO: 6 | | NO: 42 | NO: 78 | NO: 114 | NO: 150 | |
| 281 | ACATCAGTC | SEQ ID | SEQ ID | | SEQ ID | SEQ ID | SEQ ID | 4591-1 |
| | AGAGACG.HC | NO: 7 | NO: 25 | | NO: 61 | NO: 97 | NO: 133 | |
| 282 | ACATCAGTC | SEQ ID | | SEQ ID | SEQ ID | SEQ ID | SEQ ID | 4591-1 |
| | AGAGACG.LC | NO: 7 | | NO: 43 | NO: 79 | NO: 115 | NO: 151 | |
| 8711 | GGAAAGCGT | SEQ ID | SEQ ID | | SEQ ID | SEQ ID | SEQ ID | 4513-17 |
| | TGGTTTG.HC | NO: 8 | NO: 26 | | NO: 62 | NO: 98 | NO: 134 | |
| 8712 | GGAAAGCGT | SEQ ID | | SEQ ID | SEQ ID | SEQ ID | SEQ ID | 4513-17 |
| | TGGTTTG.LC | NO: 8 | | NO: 44 | NO: 80 | NO: 116 | NO: 152 | |
| 10149 | TCGAGGCAG | SEQ ID | SEQ ID | | SEQ ID | SEQ ID | SEQ ID | 4513-16 |
| | ACTACAA.HC | NO: 9 | NO: 27 | | NO: 63 | NO: 99 | NO: 135 | |
| 10150 | TCGAGGCAG | SEQ ID | | SEQ ID | SEQ ID | SEQ ID | SEQ ID | 4513-16 |
| | ACTACAA.LC | NO: 9 | | NO: 45 | NO: 81 | NO: 117 | NO: 153 | |
| 6171 | CATCCACCA | SEQ ID | SEQ ID | | SEQ ID | SEQ ID | SEQ ID | 4513-15 |
| | GCGTCCA.HC | NO: 10 | NO: 28 | | NO: 64 | NO: 100 | NO: 136 | |
| 6172 | CATCCACCA | SEQ ID | | SEQ ID | SEQ ID | SEQ ID | SEQ ID | 4513-15 |
| | GCGTCCA.LC | NO: 10 | | NO: 46 | NO: 82 | NO: 118 | NO: 154 | |
| 4185 | AACTCAGAG | SEQ ID | SEQ ID | | SEQ ID | SEQ ID | SEQ ID | 4513-14 |
| | AGTTGGC.HC | NO: 11 | NO: 29 | | NO: 65 | NO: 101 | NO: 137 | |
| 4186 | AACTCAGAG | SEQ ID | | SEQ ID | SEQ ID | SEQ ID | SEQ ID | 4513-14 |
| | AGTTGGC.LC | NO: 11 | | NO: 47 | NO: 83 | NO: 119 | NO: 155 | |
| 5365 | ATCATGGGT | SEQ ID | SEQ ID | | SEQ ID | SEQ ID | SEQ ID | 4513-12 |
| | AGATTAG.HC | NO: 12 | NO: 30 | | NO: 66 | NO: 102 | NO: 138 | |
| 5366 | ATCATGGGT | SEQ ID | | SEQ ID | SEQ ID | SEQ ID | SEQ ID | 4513-12 |
| | AGATTAG.LC | NO: 12 | | NO: 48 | NO: 84 | NO: 120 | NO: 156 | |
| 6679 | CCTTCGATC | SEQ ID | SEQ ID | | SEQ ID | SEQ ID | SEQ ID | 4513-11 |
| | TAAGCCA.HC | NO: 13 | NO: 31 | | NO: 67 | NO: 103 | NO: 139 | |
| 6680 | CCTTCGATC | SEQ ID | | SEQ ID | SEQ ID | SEQ ID | SEQ ID | 4513-11 |
| | TAAGCCA.LC | NO: 13 | | NO: 49 | NO: 85 | NO: 121 | NO: 157 | |
| 11755 | TTGGCAAAG | SEQ ID | SEQ ID | | SEQ ID | SEQ ID | SEQ ID | 4487-5 |
| | AATTCCC.HC | NO: 14 | NO: 32 | | NO: 68 | NO: 104 | NO: 140 | |
| 11756 | TTGGCAAAG | SEQ ID | | SEQ ID | SEQ ID | SEQ ID | SEQ ID | 4487-5 |
| | AATTCCC.LC | NO: 14 | | NO: 50 | NO: 86 | NO: 122 | NO: 158 | |
| 11585 | GCAGCCAGT | SEQ ID | SEQ ID | | SEQ ID | SEQ ID | SEQ ID | 4487-4 |
| | AAAGGAG.HC | NO: 15 | NO: 33 | | NO: 69 | NO: 105 | NO: 141 | |
| 11586 | GCAGCCAGT | SEQ ID | | SEQ ID | SEQ ID | SEQ ID | SEQ ID | 4487-4 |
| | AAAGGAG.LC | NO: 15 | | NO: 51 | NO: 87 | NO: 123 | NO: 159 | |
| 11489 | CGGCTAGTC | SEQ ID | SEQ ID | | SEQ ID | SEQ ID | SEQ ID | 4487-3 |
| | TTTAGTC.HC | NO: 16 | NO: 34 | | NO: 70 | NO: 106 | NO: 142 | |
| 11490 | CGGCTAGTC | SEQ ID | | SEQ ID | SEQ ID | SEQ ID | SEQ ID | 4487-3 |
| | TTTAGTC.LC | NO: 16 | | NO: 52 | NO: 88 | NO: 124 | NO: 160 | |
| 11637 | GTGCATAAG | SEQ ID | SEQ ID | | SEQ ID | SEQ ID | SEQ ID | 4487-2 |
| | AATCTCC.HC | NO: 17 | NO: 35 | | NO: 71 | NO: 107 | NO: 143 | |
| 11638 | GTGCATAAG | SEQ ID | | SEQ ID | SEQ ID | SEQ ID | SEQ ID | 4487-2 |
| | AATCTCC.LC | NO: 17 | | NO: 53 | NO: 89 | NO: 125 | NO: 161 | |
| 11495 | CGTCACTGT | SEQ ID | SEQ ID | | SEQ ID | SEQ ID | SEQ ID | 4487-1 |
| | AATAGCA.HC | NO: 18 | NO: 36 | | NO: 72 | NO: 108 | NO: 144 | |
| 11496 | CGTCACTGT | SEQ ID | | SEQ ID | SEQ ID | SEQ ID | SEQ ID | 4487-1 |
| | AATAGCA.LC | NO: 18 | | NO: 54 | NO: 90 | NO: 126 | NO: 162 | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES
SEQ ID NO: 1
ACGTCAAAGTACGCGA
SEQ ID NO: 2
CTACACCTCCAAATGC
SEQ ID NO: 3
AGCATACTCACGGTTA
SEQ ID NO: 4
ATCTACTGTCGGCACT
SEQ ID NO: 5
TAAACCGGTTATCACG
SEQ ID NO: 6
CCGTACTAGCGAAGGG
SEQ ID NO: 7
ACATCAGTCAGAGACG
SEQ ID NO: 8
GGAAAGCGTTGGTTTG
SEQ ID NO: 9
TCGAGGCAGACTACAA
SEQ ID NO: 10
CATCCACCAGCGTCCA
SEQ ID NO: 11
AACTCAGAGAGTTGGC
SEQ ID NO: 12
ATCATGGGTAGATTAG
SEQ ID NO: 13
CCTTCGATCTAAGCCA
SEQ ID NO: 14
TTGGCAAAGAATTCCC
SEQ ID NO: 15
GCAGCCAGTAAAGGAG
SEQ ID NO: 16
CGGCTAGTCTTTAGTC
SEQ ID NO: 17
GTGCATAAGAATCTCC
SEQ ID NO: 18
CGTCACTGTAATAGCA
SEQ ID NO: 19
EVQLVESGGGLVRPGGSLRLSCAASGFTFNTYEMNWVRQAPGKGLEWLSY
ISSTGNNIFYADSVKGRFTISRDNAKNSLYLQMNNVRTDDTALYYCARGG
QLWFGERLNFQHWGQGTQVTVSS
SEQ ID NO: 20
EVQLVESGGGLVQPGGSLRLSCAASGFTFNTYEMNWVRQAPGKGLEWVSY
ISGTGNNIYYADSVKGRFTISRDNAKNSLYLQMDNLRAEDTAVYYCARGG
QLWFGERLYFHFWGQGTLVAVSS
SEQ ID NO: 21
EVQLVESGGDLVQPGGSLRLSCLGSGFDFRTYGMNWVRQAPGKGLEWVSY
ISSTSSAIYYTDSIEGRFTISRDNGKNLLSLQMSSLRDEDTAVYYCARGG
SLWFGREFHAMDVWGRGTTVTVSS
SEQ ID NO: 22
EVQLVESGGGLVQPGGSLRLSCAASGFDFRTYSMNWVRQAPGKGLEWLSY
ISSTSATIYSADSVKGRFTISRDNAKNSVYLQMNSLRVDDTAVYFCARER
ITVFFRGRSQHASNVWGQGTMVTVSS
SEQ ID NO: 23
EVQLVESGGALVQPGGSLRLSCAASGFNFISYGMNWVRQAPGKGLEWISY
ISSASNTIYYADSVKGRFTISRDNAKNSLYLQMTSLRDGDSALYYCARDI
VTIFFRGRSQHAFSVWGQGTAVAVSS
SEQ ID NO: 24
QVQLQQWGAGLLKPSETLSLTCDVSGGSFSDFYWSWVRQSPGKGLEWIGD
INHSGSSNYIPSLDGRVTISVDTSRSQFSLNLTSVTAADTAIYFCARKVW
PFNGMDVWSQGTTVTVSS
SEQ ID NO: 25
QLQMQESGPRLVKPSETLSLTCAVFGDPNTISTHNWNWFRQSPGKGLEWL
GAIYYIQEIHYNPSLGSRVTMSLDAPRDQFSLTLKSVTAADTGIYYCASE
PVKIFGVVSLGEVFYVGMDVWGQGITVTVAS
SEQ ID NO: 26
QVHLVQSGAEVKKPGSSVRVSCTASGGTFHTYTVNWLRQAPGQGLEWIGG
IIPVFATENFAPKFQGRVTIKMDASTGSAYMDLASLISDDTAIYFCARGR
HSVVEPAAKYRFDTWGPGTLVVVSD
SEQ ID NO: 27
EVQLLESGGGLVQPGGSRRLSCAASGFTFSTNAMSWVRQAPGKGLEWVST
ISGSRDRTYNADSVKGRFTISRDNSKNTLYLEMNSLRAEDTAVYYCARDI
GLGRGHDYWGQGTLVTVSS
SEQ ID NO: 28
EVQLVESGGGLVQPGGSLRLSCAASGFTFSSYEMNWVRQAPGKGLEWVSY
ISSSGSTIYYADSVKGRFTISRDNAKNSLYLQMNSLRAEDTAVYYCARDF
YYDSSGYYPDAFDIWGQGTMVTVSS
SEQ ID NO: 29
QVQLVQSGREVKKFGSSVRVSCKASGGSFDTYAISWVRQAPGQGLEWMGQ
ITPMFGTSHMSQRFQGRLTIDTDDSTGTVFMDMRGLRTEDSATYYCAKDW
SFDDDGGAYVPSAMDVWGQGTTVIVSS
SEQ ID NO: 30
QLQLQESGPGLVKPSETLALTCSVSGGSIFSSNYYWAWIRQPPGKGLEWM
GSVYFSGTTFYNPSLKSRVIVSVDTAKNQFSLKLNSVSAADTAIYYCARD
VRMRSGRPRQYCMDVWGKGTTVTVSS
SEQ ID NO: 31
QVQLAQSGAEVKKPGSSVKVSCHVSGGTFSTYAINWVRQAPGHGPEWMGR
ITPISGATDYAQKFQARITISADESTSAAYLEVRTLRSDDTAVYYCARVG
QPKFFERSSDGEHFQHWGQGTLVTVSS
SEQ ID NO: 32
QMQLVQSGPEVRKPGSSVKVSCKASGGTFDNSAITWVRQAPGQGLEWMGG
IIPFVGAAAYAQRFLGRATITADASTTSSYLELRGLTSDDTGVYFCARDG
LRLNFYYYGMDVWGQGTPIIVSS
SEQ ID NO: 33
QVQLVQSGTELTKPGMSVKISCRASGTTFTRPYVHWVRQAPGQGLEWMGM
INPTGVNVFSAQKFQGRVTMTRQTSTHTSFLELVSLTGDDTAVYYCATSS
GVVSLRSAFDIWGQGTILIVSS
SEQ ID NO: 34
QITLRESGPTVVKPTQTLTLTCSFSGFSLTTTGVGVAWVRQPPGKALEWL
ALIYWDDRARYSPSLEGRLTITKDTSENQVVLRMTNMDPVDTATYYCAYR
LEHPSNWYDYFHHWGQGTLVTVSS
SEQ ID NO: 35
QVQLQESGPGLVKPSETLSLTCTVSGGSMNNYYWSWIRQTPRNGLEWIAY
ISSGGTTNYNPSLKSRVTISADTSKNQFSLGLSAATAADTALYYCARMFY
ESSTGYPSWFDPWGQGILVTVSS
SEQ ID NO: 36
QVQLVESGGGVVQPGKSLRLSCAASGFTFSSYAMHWVRQAPGKGLDWVAI
IWFDGSSKYYADSVKGRFTISRDNSKNTLYLQMNSLRADDTAVYYCARDP
AAGRSYYYYYVDVWGRGTTVTVSS
SEQ ID NO: 37
DIQMTQSPSSLSASVGDRVTITCRASQTISNNLNWYQQKPGKAPKFLIYS
VSVLQSGVPSRFSGSGSGTDFTLTITSLQPEDFATYYCQQSHSTPLTFGQ
GTRLEIK
SEQ ID NO: 38
DIQMSQSPSSLSASVGDRVTITCRASQSIGNNLNWYQQKPGKAPNLLIYA
ASNLQSGVPSRFSGSGSGTDFSLTISSLQPEDFTIYYCQQSFSTPITFGQ
GTRLEIK
SEQ ID NO: 39
QAVVTQEPSLTVSPGGTVTLTCGSSTGAVTSDHYPYWFQQKPGQAPRTLI
YDTINRHSWAPARFSASLLGGKAALTLSGAQPEDEAEYYCLLSYSGARRV
FGGGTKLTVL
SEQ ID NO: 40
SYVLTQAPSVSVAPGQTARITCGGDNIGNKNAHWYQQKPGQAPVLVVYDN
SDRPSGIPERFSGSNSGNTATLTISRVEAGDEADYFCQVWDSSSDHQVFG
GGTKLTVL
SEQ ID NO: 41
SYVLTQPPSVSVAPGQTARITCGGNNIGSKNVHWYQQKPGQAPVLVVYDN
SDRPSGVPERFSGSNSGNTATLTITRVEAGDEADYYCQVWDISSDHQVFG
GGTKLTAL
SEQ ID NO: 42
ATQLTQSPSFLSASVGDRVTITCRASQGIRNFLAWYQQKPGKAPNLLIYA
ASTLQSGVPSRFAGSGSGTEFTLTINNVQPEDFATYYCQQLTNYATFGQG
TKLEIQ
SEQ ID NO: 43
SNELKQPPSVSVSPGQTARIACSGDVMPPRSVNWYQQKAGRAPVILTFRD
TERSSGIPQRFSASRSGTTVTLTINGVQADDEADYYCQSTDMNSIYKTVG
GGTRLTVL
SEQ ID NO: 44
DIQMTQSPSSLSASVGDGVTITCRSSQSISTHLNWYQQKPGMAPKLLLYS
SSTLQGGVPSRFSGSGSGTDFTLTISGLQPEDFATYYCQQSYSTPPTFGG
GTKVDIK
SEQ ID NO: 45
QSALTQPASVSGSPGQSVTISCTGTSSDVGTYNYVSWYQQHPGKAPKLMI
YEVSNRPSGVSNRFSGSKSGNTASLTISGLQAEDEADYYCSSYIAYNILE
VFGGGTKLTVL
SEQ ID NO: 46
EIVMTQSPATLSVSPGERATLSCRASQSVSSNLAWYQQKPGQAPRLLIYG
ASTRATGIPARFSGSGSGTEFTLTISSLQSEDFAVYYCQQYNNGRTFGQG
TKVEIK
SEQ ID NO: 47
EIVLTQSPGTLSVSPGERATLSCKASQSISSSSLAWYRQTPGQTPTLLIY
GTSTRATGIPDRFSGRGSGTDFTLTITNVKPEDFAVYYCQQYGSSPFTFG
PGTRLDFR
SEQ ID NO: 48
EVVLTQSPGTLSLSPGERATLSCRASQSVNSIYLAWYQQKPGQAPRLLIY
GASNRAAGIPDRFSGSGSGTDFTLTIDRLEPEDFAVYYCQQYDTSPAVTF
GPGTKVEIK
SEQ ID NO: 49
DIQMTQSPSTLSASVGDRTTITCRASQNIDKWLAWYQQKPGKAPTVLIFK
ASTLESGVPSRFSGSSSGTEFTLTIDNLQPEDSATYFCHQYSRYPWTFGQ
GTKVELK
SEQ ID NO: 50
QIVLTQSPGTLSLSPGDTATLSCRASQRVTHTHLAWYQQKPGQPPRLLIY
ATSQRATGIPDRFRGSGSGRDFTLTIASLEPEDFAVYYCQQNGESPRMFG
QGTKLDVK
SEQ ID NO: 51
QSALSQPASVSGSLGQSITISCSGVGSDAVTYTVVSWYQQHPTKAPKIII
SDGDKRPSGVSSRFSGSKSGNTAFLRISGLQTDDEALYYCCSYVGSNTWV
FGGGTTVTVL
SEQ ID NO: 52
DIQMTQSPSTLSASVGDTVTITCRASQNIHSWLAWYQQKPGQAPKLLIYQ
ASTLQSGVPSRFRGSGSGTEFTLTISSLQPDDFATYHCQQYNFHSPTFGQ
GTKVEIK
SEQ ID NO: 53
AIELTQSPESLAVSPGERATINCKSSQNILYSSNNKNYLAWYQQKVGQPP
ELLIYWASTRKSGVPDRFSGGGSGTDFTLTISSLQADDVALYYCQQYFYT
PWTFGQGTKVEIK
SEQ ID NO: 54
QSVLTQPPSVSGGPGQRVTISCTGSTSNIGAGYDVHWYQQLPGTAPKLLI
YGNNNRPSGVPDRFSGSKSGTSASLAITGLQAVDEADYYCQSYDSNLSGV
VFGGGTKLTVL
SEQ ID NO: 55
GFTFNTYE
SEQ ID NO: 56
GFTFNTYE
SEQ ID NO: 57
GFDFRTYG
SEQ ID NO: 58
GFDFRTYS
SEQ ID NO: 59
GFNFISYG
SEQ ID NO: 60
GGSFSDFY
SEQ ID NO: 61
GDPNTISTHN
SEQ ID NO: 62
GGTFHTYT
SEQ ID NO: 63
GFTFSTNA
SEQ ID NO: 64
GFTFSSYE
SEQ ID NO: 65
GGSFDTYA
SEQ ID NO: 66
GGSIFSSNYY
SEQ ID NO: 67
GGTFSTYA
SEQ ID NO: 68
GGTFDNSA
SEQ ID NO: 69
GTTFTRPY
SEQ ID NO: 70
GFSLTTTGVG
SEQ ID NO: 71
GGSMNNYY
SEQ ID NO: 72
GFTFSSYA
SEQ ID NO: 73
QTISNN
SEQ ID NO: 74
QSIGNN
SEQ ID NO: 75
TGAVTSDHY
SEQ ID NO: 76
NIGNKN
SEQ ID NO: 77
NIGSKN
SEQ ID NO: 78
QGIRNF
SEQ ID NO: 79
VMPPRS
SEQ ID NO: 80
QSISTH
SEQ ID NO: 81
SSDVGTYNY
SEQ ID NO: 82
QSVSSN
SEQ ID NO: 83
QSISSSS
SEQ ID NO: 84
QSVNSIY
SEQ ID NO: 85
QNIDKW
SEQ ID NO: 86
QRVTHTH
SEQ ID NO: 87
GSDAVTYTV
SEQ ID NO: 88
QNIHSW
SEQ ID NO: 89
QNILYSSNNKNY
SEQ ID NO: 90
TSNIGAGYD
SEQ ID NO: 91
ISSTGNNI
SEQ ID NO: 92
ISGTGNNI
SEQ ID NO: 93
ISSTSSAI
SEQ ID NO: 94
ISSTSATI
SEQ ID NO: 95
ISSASNTI
SEQ ID NO: 96
INHSGSS
SEQ ID NO: 97
IYYIQEI
SEQ ID NO: 98
IIPVFATE
SEQ ID NO: 99
ISGSRDRT
SEQ ID NO: 100
ISSSGSTI
SEQ ID NO: 101
ITPMFGTS
SEQ ID NO: 102
VYFSGTT
SEQ ID NO: 103
ITPISGAT
SEQ ID NO: 104
IIPFVGAA
SEQ ID NO: 105
INPTGVNV
SEQ ID NO: 106
IYWDDRA
SEQ ID NO: 107
ISSGGTT
SEQ ID NO: 108
IWFDGSSK
SEQ ID NO: 109
SVS
SEQ ID NO: 110
AAS
SEQ ID NO: 111
DTI
SEQ ID NO: 112
DNS
SEQ ID NO: 113
DNS
SEQ ID NO: 114
AAS
SEQ ID NO: 115
RDT
SEQ ID NO: 116
SSS
SEQ ID NO: 117
EVS
SEQ ID NO: 118
GAS
SEQ ID NO: 119
GTS
SEQ ID NO: 120
GAS
SEQ ID NO: 121
KAS
SEQ ID NO: 122
ATS
SEQ ID NO: 123
DGD
SEQ ID NO: 124
QAS
SEQ ID NO: 125
WAS
SEQ ID NO: 126
GNN
SEQ ID NO: 127
ARGGQLWFGERLNFQH
SEQ ID NO: 128
ARGGQLWFGERLYFHF
SEQ ID NO: 129
ARGGSLWFGREFHAMDV
SEQ ID NO: 130
ARERITVFFRGRSQHASNV
SEQ ID NO: 131
ARDIVTIFFRGRSQHAFSV
SEQ ID NO: 132
ARKVWPFNGMDV
SEQ ID NO: 133
ASEPVKIFGVVSLGEVFYVGMDV
SEQ ID NO: 134
ARGRHSVVEPAAKYRFDT
SEQ ID NO: 135
ARDIGLGRGHDY
SEQ ID NO: 136
ARDFYYDSSGYYPDAFDI
SEQ ID NO: 137
AKDWSFDDDGGAYVPSAMDV
SEQ ID NO: 138
ARDVRMRSGRPRQYCMDV
SEQ ID NO: 139
ARVGQPKFFERSSDGEHFQH
SEQ ID NO: 140
ARDGLRLNFYYYGMDV
SEQ ID NO: 141
ATSSGVVSLRSAFDI
SEQ ID NO: 142
AYRLEHPSNWYDYFHH
SEQ ID NO: 143
ARMFYESSTGYPSWFDP
SEQ ID NO: 144
ARDPAAGRSYYYYYVDV
SEQ ID NO: 145
QQSHSTPLT
SEQ ID NO: 146
QQSFSTPIT
SEQ ID NO: 147
LLSYSGARRV
SEQ ID NO: 148
QVWDSSSDHQV
SEQ ID NO: 149
QVWDISSDHQV
SEQ ID NO: 150
QQLTNYAT
SEQ ID NO: 151
QSTDMNSIYKT
SEQ ID NO: 152
QQSYSTPPT
SEQ ID NO: 153
SSYIAYNILEV
SEQ ID NO: 154
QQYNNGRT
SEQ ID NO: 155
QQYGSSPFT
SEQ ID NO: 156
QQYDTSPAVT
SEQ ID NO: 157
HQYSRYPWT
SEQ ID NO: 158
QQNGESPRM
SEQ ID NO: 159
CSYVGSNTWV
SEQ ID NO: 160
QQYNFHSPT
SEQ ID NO: 161
QQYFYTPWT
SEQ ID NO: 162
QSYDSNLSGVV
SEQ ID NO: 163
CARGGSLWFGREFHAMDVW
SEQ ID NO: 164
CARGGQLWFGERLYFHFW
SEQ ID NO: 165
CARGGQLWFGERLNFQHW
SEQ ID NO: 166
CARDIVTIFFRGRSQHAFSVW
SEQ ID NO: 167
CARERITVFFRGRSQHASNVW
SEQ ID NO: 168
CARKVWPFNGMDVW
SEQ ID NO: 169
CASEPVKIFGVVSLGEVFYVGMDVW
SEQ ID NO: 170
CLLSYSGARRVF
SEQ ID NO: 171
CQQSFSTPITF
SEQ ID NO: 172
CQQSHSTPLTF
SEQ ID NO: 173
CQVWDISSDHQVF
SEQ ID NO: 174
CQVWDSSSDHQVF
SEQ ID NO: 175
CQQLTNYATF
SEQ ID NO: 176
QSTDMNSIYKT
SEQ ID NO: 177
CARDPAAGRSYYYYYVDVW
SEQ ID NO: 178
CARMFYESSTGYPSWFDPW
SEQ ID NO: 179
CAYRLEHPSNWYDYFHHW
SEQ ID NO: 180
CATSSGVVSLRSAFDIW
SEQ ID NO: 181
CARDGLRLNFYYYGMDVW
SEQ ID NO: 182
CARVGQPKFFERSSDGEHFQHW
SEQ ID NO: 183
CARDVRMRSGRPRQYCMDVW
SEQ ID NO: 184
CAKDWSFDDDGGAYVPSAMDVW
SEQ ID NO: 185
CARDFYYDSSGYYPDAFDIW
SEQ ID NO: 186
CARDIGLGRGHDYW
SEQ ID NO: 187
CARGRHSVVEPAAKYRFDTW
SEQ ID NO: 188
CQSYDSNLSGVVF
SEQ ID NO: 189
CQQYFYTPWTF
SEQ ID NO: 190
CQQYNFHSPTF
SEQ ID NO: 191
CCSYVGSNTWVF
SEQ ID NO: 192
CQQNGESPRMF
SEQ ID NO: 193
CHQYSRYPWTF
SEQ ID NO: 194
CQQYDTSPAVTF
SEQ ID NO: 195
CQQYGSSPFTF
SEQ ID NO: 196
CQQYNNGRTF
SEQ ID NO: 197
CSSYIAYNILEVF
SEQ ID NO: 198
CQQSYSTPPTF
SEQ ID NO: 199
ATTCGCCTTACGCAA
SEQ ID NO: 200
AACCCACCGTTGTTA
SEQ ID NO: 201
GGTAGCCCTAGAGTA
SEQ ID NO: 202
CAGTAAGTTCGGGAC
SEQ ID NO: 203
CTTCACTCTGTCAGG
SEQ ID NO: 204
TACGCCTATAACTTG
SEQ ID NO: 205
AGACTAATAGCTGAC
SEQ ID NO: 206
GCTCCTTTACACGTA
SEQ ID NO: 207
GCAGCGTATAAGTCA
SEQ ID NO: 208
GCTCCTTTACACGTA
SEQ ID NO: 209
CTTCACTCTGTCAGG
SEQ ID NO: 210
TGGTAACGACAGTCC
SEQ ID NO: 211
TGTGTATTCCCTTGT
SEQ ID NO: 212
TACGCCTATAACTTG
SEQ ID NO: 213
GTGTGTTGTCCTATG
SEQ ID NO: 214
AACCCACCGTTGTTA
SEQ ID NO: 215
CAGTAAGTTCGGGAC
SEQ ID NO: 216
GTAAGACGCCTATGC
SEQ ID NO: 217
TTTCAACGCCCTTTC
SEQ ID NO: 218
CCGTCCTGATAGATG
SEQ ID NO: 219
TCATTTCCTCCGATT
SEQ ID NO: 220
GGTAGCCCTAGAGTA
SEQ ID NO: 221
AGACTAATAGCTGAC
SEQ ID NO: 222
CAGCCCACTGCAATA
SEQ ID NO: 223
ATCGTCGAGAGCTAG
SEQ ID NO: 224
TCACAGTTCCTTGGA
SEQ ID NO: 225
CAGATGATCCACCAT
SEQ ID NO: 226
GACCTCATTGTGAAT
SEQ ID NO: 227
TGACCTTCCTCTCCT
SEQ ID NO: 228
CAGGTCCCTTATTTC
SEQ ID NO: 229
ACAATTTGTCTGCGA
SEQ ID NO: 230
AACCTTCCGTCTAAG
SEQ ID NO: 231
AATCACGGTCCTTGT
SEQ ID NO: 232
CCTTGGCACCCGAGAATTCCANNNNNNNNNNNNNCCCATATAAGAAA

What is claimed is:

1. A recombinant antibody, wherein the antibody comprises a light chain variable region (VL) that comprises a light chain complementarity determining region (CDRL)1, CDRL2, and CDRL3 and/or a heavy chain variable region (VH) that comprises a heavy chain complementarity determining region (CDRH)1, CDRH2, and CDRH3, wherein:
the CDRH3 comprises an amino acid sequence at least 60% identical to SEQ ID NOs: 127-144; and
the CDRL3 comprises an amino acid sequence at least 60% identical to SEQ ID NOs: 145-162.

2. The recombinant antibody of claim 1, wherein the CDRH3 comprises at least one amino acid substitution when compared to SEQ ID NOs: 127-144.

3. The recombinant antibody of claim 1, wherein the CDRL3 comprises at least one amino acid substitution when compared to SEQ ID NOs: SEQ ID NOs: 145-162.

4. The recombinant antibody of claim 1, wherein:
the CDRH1 comprises an amino acid sequence at least 60% identical to SEQ ID NOs: 55-72; and/or the CDRL1 comprises an amino acid sequence at least 60% identical to SEQ ID NOs: 73-90.

5. The recombinant antibody of claim 1, wherein:
the CDRH2 comprises an amino acid sequence at least 60% identical to SEQ ID NOs: 91-108; and/or
the CDRL2 comprises an amino acid sequence at least 60% identical to SEQ ID NOs: 109-126.

6. The recombinant antibody of claim 1, wherein the VH comprises an amino acid sequence selected from the group consisting of SEQ ID NOs: 1-36.

7. The recombinant antibody of claim 1, wherein the VL comprises an amino acid sequence selected from the group consisting of SEQ ID NOs: 37-54.

8. The recombinant antibody of claim 1, wherein
the CDRH1 comprises the amino acid sequence of SEQ ID NO: 55,
the CDRH2 comprises the amino acid sequence of SEQ ID NO: 91,
the CDRH3 comprises the amino acid sequence of SEQ ID NO: 127,
the CDRL1 comprises the amino acid sequence of SEQ ID NO: 73,
the CDRL2 comprises the amino acid sequence of SEQ ID NO: 109, and
the CDRL3 comprises the amino acid sequence of SEQ ID NO: 145; or
the CDRH1 comprises the amino acid sequence of SEQ ID NO: 56,
the CDRH2 comprises the amino acid sequence of SEQ ID NO: 92,
the CDRH3 comprises the amino acid sequence of SEQ ID NO: 128,
the CDRL1 comprises the amino acid sequence of SEQ ID NO: 74,
the CDRL2 comprises the amino acid sequence of SEQ ID NO: 110, and
the CDRL3 comprises the amino acid sequence of SEQ ID NO: 146; or
the CDRH1 comprises the amino acid sequence of SEQ ID NO: 57,
the CDRH2 comprises the amino acid sequence of SEQ ID NO: 93,
the CDRH3 comprises the amino acid sequence of SEQ ID NO: 129,
the CDRL1 comprises the amino acid sequence of SEQ ID NO: 75,
the CDRL2 comprises the amino acid sequence of SEQ ID NO: 111, and
the CDRL3 comprises the amino acid sequence of SEQ ID NO: 147; or
the CDRH1 comprises the amino acid sequence of SEQ ID NO: 58,
the CDRH2 comprises the amino acid sequence of SEQ ID NO: 94,
the CDRH3 comprises the amino acid sequence of SEQ ID NO: 130,
the CDRL1 comprises the amino acid sequence of SEQ ID NO: 76,
the CDRL2 comprises the amino acid sequence of SEQ ID NO: 112, and
the CDRL3 comprises the amino acid sequence of SEQ ID NO: 148; or
the CDRH1 comprises the amino acid sequence of SEQ ID NO: 59,
the CDRH2 comprises the amino acid sequence of SEQ ID NO: 95,
the CDRH3 comprises the amino acid sequence of SEQ ID NO: 131,
the CDRL1 comprises the amino acid sequence of SEQ ID NO: 77,
the CDRL2 comprises the amino acid sequence of SEQ ID NO: 113, and
the CDRL3 comprises the amino acid sequence of SEQ ID NO: 149; or
the CDRH1 comprises the amino acid sequence of SEQ ID NO: 60,
the CDRH2 comprises the amino acid sequence of SEQ ID NO: 96,
the CDRH3 comprises the amino acid sequence of SEQ ID NO: 132,
the CDRL1 comprises the amino acid sequence of SEQ ID NO: 78,
the CDRL2 comprises the amino acid sequence of SEQ ID NO: 114, and
the CDRL3 comprises the amino acid sequence of SEQ ID NO: 150; or
the CDRH1 comprises the amino acid sequence of SEQ ID NO: 61,
the CDRH2 comprises the amino acid sequence of SEQ ID NO: 97,
the CDRH3 comprises the amino acid sequence of SEQ ID NO: 133,
the CDRL1 comprises the amino acid sequence of SEQ ID NO: 79,
the CDRL2 comprises the amino acid sequence of SEQ ID NO: 115, and
the CDRL3 comprises the amino acid sequence of SEQ ID NO: 151; or
the CDRH1 comprises the amino acid sequence of SEQ ID NO: 62,
the CDRH2 comprises the amino acid sequence of SEQ ID NO: 98,
the CDRH3 comprises the amino acid sequence of SEQ ID NO: 134,
the CDRL1 comprises the amino acid sequence of SEQ ID NO: 80,
the CDRL2 comprises the amino acid sequence of SEQ ID NO: 116, and
the CDRL3 comprises the amino acid sequence of SEQ ID NO: 152; or
the CDRH1 comprises the amino acid sequence of SEQ ID NO: 63,
the CDRH2 comprises the amino acid sequence of SEQ ID NO: 99,
the CDRH3 comprises the amino acid sequence of SEQ ID NO: 135,
the CDRL1 comprises the amino acid sequence of SEQ ID NO: 81,
the CDRL2 comprises the amino acid sequence of SEQ ID NO: 117, and
the CDRL3 comprises the amino acid sequence of SEQ ID NO: 153; or
the CDRH1 comprises the amino acid sequence of SEQ ID NO: 64,
the CDRH2 comprises the amino acid sequence of SEQ ID NO: 100,
the CDRH3 comprises the amino acid sequence of SEQ ID NO: 136,
the CDRL1 comprises the amino acid sequence of SEQ ID NO: 82,
the CDRL2 comprises the amino acid sequence of SEQ ID NO: 118, and
the CDRL3 comprises the amino acid sequence of SEQ ID NO: 154; or
the CDRH1 comprises the amino acid sequence of SEQ ID NO: 65,
the CDRH2 comprises the amino acid sequence of SEQ ID NO: 101,
the CDRH3 comprises the amino acid sequence of SEQ ID NO: 137,
the CDRL1 comprises the amino acid sequence of SEQ ID NO: 83,
the CDRL2 comprises the amino acid sequence of SEQ ID NO: 119, and
the CDRL3 comprises the amino acid sequence of SEQ ID NO: 155; or
the CDRH1 comprises the amino acid sequence of SEQ ID NO: 66,
the CDRH2 comprises the amino acid sequence of SEQ ID NO: 102,
the CDRH3 comprises the amino acid sequence of SEQ ID NO: 138,
the CDRL1 comprises the amino acid sequence of SEQ ID NO: 84,
the CDRL2 comprises the amino acid sequence of SEQ ID NO: 120, and
the CDRL3 comprises the amino acid sequence of SEQ ID NO: 156; or
the CDRH1 comprises the amino acid sequence of SEQ ID NO: 67,
the CDRH2 comprises the amino acid sequence of SEQ ID NO: 103,
the CDRH3 comprises the amino acid sequence of SEQ ID NO: 139,
the CDRL1 comprises the amino acid sequence of SEQ ID NO: 85,
the CDRL2 comprises the amino acid sequence of SEQ ID NO: 121, and
the CDRL3 comprises the amino acid sequence of SEQ ID NO: 157; or
the CDRH1 comprises the amino acid sequence of SEQ ID NO: 68,
the CDRH2 comprises the amino acid sequence of SEQ ID NO: 104,
the CDRH3 comprises the amino acid sequence of SEQ ID NO: 140,
the CDRL1 comprises the amino acid sequence of SEQ ID NO: 86,
the CDRL2 comprises the amino acid sequence of SEQ ID NO: 122, and
the CDRL3 comprises the amino acid sequence of SEQ ID NO: 158; or
the CDRH1 comprises the amino acid sequence of SEQ ID NO: 69,
the CDRH2 comprises the amino acid sequence of SEQ ID NO: 105,
the CDRH3 comprises the amino acid sequence of SEQ ID NO: 141,
the CDRL1 comprises the amino acid sequence of SEQ ID NO: 87,
the CDRL2 comprises the amino acid sequence of SEQ ID NO: 123, and
the CDRL3 comprises the amino acid sequence of SEQ ID NO: 159; or
the CDRH1 comprises the amino acid sequence of SEQ ID NO: 70,
the CDRH2 comprises the amino acid sequence of SEQ ID NO: 106,
the CDRH3 comprises the amino acid sequence of SEQ ID NO: 142,
the CDRL1 comprises the amino acid sequence of SEQ ID NO: 88,
the CDRL2 comprises the amino acid sequence of SEQ ID NO: 124, and
the CDRL3 comprises the amino acid sequence of SEQ ID NO: 160; or
the CDRH1 comprises the amino acid sequence of SEQ ID NO: 71,
the CDRH2 comprises the amino acid sequence of SEQ ID NO: 107,
the CDRH3 comprises the amino acid sequence of SEQ ID NO: 143,
the CDRL1 comprises the amino acid sequence of SEQ ID NO: 89,
the CDRL2 comprises the amino acid sequence of SEQ ID NO: 125, and
the CDRL3 comprises the amino acid sequence of SEQ ID NO: 161; or
the CDRH1 comprises the amino acid sequence of SEQ ID NO: 72,
the CDRH2 comprises the amino acid sequence of SEQ ID NO: 108,
the CDRH3 comprises the amino acid sequence of SEQ ID NO: 144,
the CDRL1 comprises the amino acid sequence of SEQ ID NO: 90,
the CDRL2 comprises the amino acid sequence of SEQ ID NO: 126, and
the CDRL3 comprises the amino acid sequence of SEQ ID NO: 162.

9. The recombinant antibody of claim 1, wherein
the VH comprises the amino acid sequence of SEQ ID NO: 19 and
the VL comprises the amino acid sequence of SEQ ID NO: 37; or
the VH comprises the amino acid sequence of SEQ ID NO: 20 and
the VL comprises the amino acid sequence of SEQ ID NO: 38; or
the VH comprises the amino acid sequence of SEQ ID NO: 21 and
the VL comprises the amino acid sequence of SEQ ID NO: 39; or
the VH comprises the amino acid sequence of SEQ ID NO: 22 and
the VL comprises the amino acid sequence of SEQ ID NO: 40; or
the VH comprises the amino acid sequence of SEQ ID NO: 23 and
the VL comprises the amino acid sequence of SEQ ID NO: 41; or
the VH comprises the amino acid sequence of SEQ ID NO: 24 and
the VL comprises the amino acid sequence of SEQ ID NO: 42; or
the VH comprises the amino acid sequence of SEQ ID NO: 25 and
the VL comprises the amino acid sequence of SEQ ID NO: 43; or
the VH comprises the amino acid sequence of SEQ ID NO: 26 and
the VL comprises the amino acid sequence of SEQ ID NO: 44; or
the VH comprises the amino acid sequence of SEQ ID NO: 27 and
the VL comprises the amino acid sequence of SEQ ID NO: 45; or
the VH comprises the amino acid sequence of SEQ ID NO: 28 and
the VL comprises the amino acid sequence of SEQ ID NO: 46; or
the VH comprises the amino acid sequence of SEQ ID NO: 29 and
the VL comprises the amino acid sequence of SEQ ID NO: 47; or
the VH comprises the amino acid sequence of SEQ ID NO: 30 and
the VL comprises the amino acid sequence of SEQ ID NO: 48; or
the VH comprises the amino acid sequence of SEQ ID NO: 31 and
the VL comprises the amino acid sequence of SEQ ID NO: 49; or
the VH comprises the amino acid sequence of SEQ ID NO: 32 and
the VL comprises the amino acid sequence of SEQ ID NO: 50; or
the VH comprises the amino acid sequence of SEQ ID NO: 33 and
the VL comprises the amino acid sequence of SEQ ID NO: 51; or
the VH comprises the amino acid sequence of SEQ ID NO: 34 and
the VL comprises the amino acid sequence of SEQ ID NO: 52; or
the VH comprises the amino acid sequence of SEQ ID NO: 35 and
the VL comprises the amino acid sequence of SEQ ID NO: 53; or
the VH comprises the amino acid sequence of SEQ ID NO: 36 and
the VL comprises the amino acid sequence of SEQ ID NO: 54.

10. A method of treating a viral infection in a subject in need thereof comprising administering to the subject a therapeutically effective amount of a recombinant antibody of claim 1.

11. The method of claim 10, wherein the subject is infected by HIV-1.


SEQUENCE LISTING (232 sequences)

SEQ ID NO: 1 - DNA, 16 bp, synthetic construct
  source 1..16: mol_type = other DNA
acgtcaaagtacgcga

SEQ ID NO: 2 - DNA, 16 bp, synthetic construct
  source 1..16: mol_type = other DNA
ctacacctccaaatgc

SEQ ID NO: 3 - DNA, 16 bp, synthetic construct
  source 1..16: mol_type = other DNA
agcatactcacggtta

SEQ ID NO: 4 - DNA, 16 bp, synthetic construct
  source 1..16: mol_type = other DNA
atctactgtcggcact

SEQ ID NO: 5 - DNA, 16 bp, synthetic construct
  source 1..16: mol_type = other DNA
taaaccggttatcacg

SEQ ID NO: 6 - DNA, 16 bp, synthetic construct
  source 1..16: mol_type = other DNA
ccgtactagcgaaggg

SEQ ID NO: 7 - DNA, 16 bp, synthetic construct
  source 1..16: mol_type = other DNA
acatcagtcagagacg

SEQ ID NO: 8 - DNA, 16 bp, synthetic construct
  source 1..16: mol_type = other DNA
ggaaagcgttggtttg

SEQ ID NO: 9 - DNA, 16 bp, synthetic construct
  source 1..16: mol_type = other DNA
tcgaggcagactacaa

SEQ ID NO: 10 - DNA, 16 bp, synthetic construct
  source 1..16: mol_type = other DNA
catccaccagcgtcca

SEQ ID NO: 11 - DNA, 16 bp, synthetic construct
  source 1..16: mol_type = other DNA
aactcagagagttggc

SEQ ID NO: 12 - DNA, 16 bp, synthetic construct
  source 1..16: mol_type = other DNA
atcatgggtagattag

SEQ ID NO: 13 - DNA, 16 bp, synthetic construct
  source 1..16: mol_type = other DNA
ccttcgatctaagcca

SEQ ID NO: 14 - DNA, 16 bp, synthetic construct
  source 1..16: mol_type = other DNA
ttggcaaagaattccc

SEQ ID NO: 15 - DNA, 16 bp, synthetic construct
  source 1..16: mol_type = other DNA
gcagccagtaaaggag

SEQ ID NO: 16 - DNA, 16 bp, synthetic construct
  source 1..16: mol_type = other DNA
cggctagtctttagtc

SEQ ID NO: 17 - DNA, 16 bp, synthetic construct
  source 1..16: mol_type = other DNA
gtgcataagaatctcc

SEQ ID NO: 18 - DNA, 16 bp, synthetic construct
  source 1..16: mol_type = other DNA
cgtcactgtaatagca

SEQ ID NO: 19 - protein, 123 aa, synthetic construct
EVQLVESGGGLVRPGGSLRLSCAASGFTFNTYEMNWVRQAPGKGLEWLSYISSTGNNIFYADSVKGRFTISRDNAKNSLYLQMNNVRTDDTALYYCARGGQLWFGERLNFQHWGQGTQVTVSS

SEQ ID NO: 20 - protein, 123 aa, synthetic construct
EVQLVESGGGLVQPGGSLRLSCAASGFTFNTYEMNWVRQAPGKGLEWVSYISGTGNNIYYADSVKGRFTISRDNAKNSLYLQMDNLRAEDTAVYYCARGGQLWFGERLYFHFWGQGTLVAVSS

SEQ ID NO: 21 - protein, 124 aa, synthetic construct
EVQLVESGGDLVQPGGSLRLSCLGSGFDFRTYGMNWVRQAPGKGLEWVSYISSTSSAIYYTDSIEGRFTISRDNGKNLLSLQMSSLRDEDTAVYYCARGGSLWFGREFHAMDVWGRGTTVTVSS

SEQ ID NO: 22 - protein, 126 aa, synthetic construct
EVQLVESGGGLVQPGGSLRLSCAASGFDFRTYSMNWVRQAPGKGLEWLSYISSTSATIYSADSVKGRFTISRDNAKNSVYLQMNSLRVDDTAVYFCARERITVFFRGRSQHASNVWGQGTMVTVSS

SEQ ID NO: 23 - protein, 126 aa, synthetic construct
EVQLVESGGALVQPGGSLRLSCAASGFNFISYGMNWVRQAPGKGLEWISYISSASNTIYYADSVKGRFTISRDNAKNSLYLQMTSLRDGDSALYYCARDIVTIFFRGRSQHAFSVWGQGTAVAVSS

SEQ ID NO: 24 - protein, 118 aa, synthetic construct
QVQLQQWGAGLLKPSETLSLTCDVSGGSFSDFYWSWVRQSPGKGLEWIGDINHSGSSNYIPSLDGRVTISVDTSRSQFSLNLTSVTAADTAIYFCARKVWPFNGMDVWSQGTTVTVSS

SEQ ID NO: 25 - protein, 131 aa, synthetic construct
QLQMQESGPRLVKPSETLSLTCAVFGDPNTISTHNWNWFRQSPGKGLEWLGAIYYIQEIHYNPSLGSRVTMSLDAPRDQFSLTLKSVTAADTGIYYCASEPVKIFGVVSLGEVFYVGMDVWGQGITVTVAS

SEQ ID NO: 26 - protein, 125 aa, synthetic construct
QVHLVQSGAEVKKPGSSVRVSCTASGGTFHTYTVNWLRQAPGQGLEWIGGIIPVFATENFAPKFQGRVTIKMDASTGSAYMDLASLISDDTAIYFCARGRHSVVEPAAKYRFDTWGPGTLVVVSD

SEQ ID NO: 27 - protein, 119 aa, synthetic construct
EVQLLESGGGLVQPGGSRRLSCAASGFTFSTNAMSWVRQAPGKGLEWVSTISGSRDRTYNADSVKGRFTISRDNSKNTLYLEMNSLRAEDTAVYYCARDIGLGRGHDYWGQGTLVTVSS

SEQ ID NO: 28 - protein, 125 aa, synthetic construct
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCARDFYYDSSGYYPDAFDIWGQGTMVTVSS

SEQ ID NO: 29 - protein, 127 aa, synthetic construct
QVQLVQSGREVKKFGSSVRVSCKASGGSFDTYAISWVRQAPGQGLEWMGQITPMFGTSHMSQRFQGRLTIDTDDSTGTVFMDMRGLRTEDSATYYCAKDWSFDDDGGAYVPSAMDVWGQGTTVIVSS

SEQ ID NO: 30 - protein, 126 aa, synthetic construct
QLQLQESGPGLVKPSETLALTCSVSGGSIFSSNYYWAWIRQPPGKGLEWMGSVYFSGTTFYNPSLKSRVIVSVDTAKNQFSLKLNSVSAADTAIYYCARDVRMRSGRPRQYCMDVWGKGTTVTVSS

SEQ ID NO: 31 - protein, 127 aa, synthetic construct
QVQLAQSGAEVKKPGSSVKVSCHVSGGTFSTYAINWVRQAPGHGPEWMGRITPISGATDYAQKFQARITISADESTSAAYLEVRTLRSDDTAVYYCARVGQPKFFERSSDGEHFQHWGQGTLVTVSS

SEQ ID NO: 32 - protein, 123 aa, synthetic construct
QMQLVQSGPEVRKPGSSVKVSCKASGGTFDNSAITWVRQAPGQGLEWMGGIIPFVGAAAYAQRFLGRATITADASTTSSYLELRGLTSDDTGVYFCARDGLRLNFYYYGMDVWGQGTPIIVSS

SEQ ID NO: 33 - protein, 122 aa, synthetic construct
QVQLVQSGTELTKPGMSVKISCRASGTTFTRPYVHWVRQAPGQGLEWMGMINPTGVNVFSAQKFQGRVTMTRQTSTHTSFLELVSLTGDDTAVYYCATSSGVVSLRSAFDIWGQGTILIVSS

SEQ ID NO: 34 - protein, 124 aa, synthetic construct
QITLRESGPTVVKPTQTLTLTCSFSGFSLTTTGVGVAWVRQPPGKALEWLALIYWDDRARYSPSLEGRLTITKDTSENQVVLRMTNMDPVDTATYYCAYRLEHPSNWYDYFHHWGQGTLVTVSS

SEQ ID NO: 35 - protein, 123 aa, synthetic construct
QVQLQESGPGLVKPSETLSLTCTVSGGSMNNYYWSWIRQTPRNGLEWIAYISSGGTTNYNPSLKSRVTISADTSKNQFSLGLSAATAADTALYYCARMFYESSTGYPSWFDPWGQGILVTVSS

SEQ ID NO: 36 - protein, 124 aa, synthetic construct
QVQLVESGGGVVQPGKSLRLSCAASGFTFSSYAMHWVRQAPGKGLDWVAIIWFDGSSKYYADSVKGRFTISRDNSKNTLYLQMNSLRADDTAVYYCARDPAAGRSYYYYYVDVWGRGTTVTVSS

SEQ ID NO: 37 - protein, 107 aa, synthetic construct
DIQMTQSPSSLSASVGDRVTITCRASQTISNNLNWYQQKPGKAPKFLIYSVSVLQSGVPSRFSGSGSGTDFTLTITSLQPEDFATYYCQQSHSTPLTFGQGTRLEIK

SEQ ID NO: 38 - protein, 107 aa, synthetic construct
DIQMSQSPSSLSASVGDRVTITCRASQSIGNNLNWYQQKPGKAPNLLIYAASNLQSGVPSRFSGSGSGTDFSLTISSLQPEDFTIYYCQQSFSTPITFGQGTRLEIK

SEQ ID NO: 39 - protein, 110 aa, synthetic construct
QAVVTQEPSLTVSPGGTVTLTCGSSTGAVTSDHYPYWFQQKPGQAPRTLIYDTINRHSWAPARFSASLLGGKAALTLSGAQPEDEAEYYCLLSYSGARRVFGGGTKLTVL

SEQ ID NO: 40 - protein, 108 aa, synthetic construct
SYVLTQAPSVSVAPGQTARITCGGDNIGNKNAHWYQQKPGQAPVLVVYDNSDRPSGIPERFSGSNSGNTATLTISRVEAGDEADYFCQVWDSSSDHQVFGGGTKLTVL

SEQ ID NO: 41 - protein, 108 aa, synthetic construct
SYVLTQPPSVSVAPGQTARITCGGNNIGSKNVHWYQQKPGQAPVLVVYDNSDRPSGVPERFSGSNSGNTATLTITRVEAGDEADYYCQVWDISSDHQVFGGGTKLTAL

SEQ ID NO: 42 - protein, 106 aa, synthetic construct
ATQLTQSPSFLSASVGDRVTITCRASQGIRNFLAWYQQKPGKAPNLLIYAASTLQSGVPSRFAGSGSGTEFTLTINNVQPEDFATYYCQQLTNYATFGQGTKLEIQ

SEQ ID NO: 43 - protein, 108 aa, synthetic construct
SNELKQPPSVSVSPGQTARIACSGDVMPPRSVNWYQQKAGRAPVILTFRDTERSSGIPQRFSASRSGTTVTLTINGVQADDEADYYCQSTDMNSIYKTVGGGTRLTVL

SEQ ID NO: 44 - protein, 107 aa, synthetic construct
DIQMTQSPSSLSASVGDGVTITCRSSQSISTHLNWYQQKPGMAPKLLLYSSSTLQGGVPSRFSGSGSGTDFTLTISGLQPEDFATYYCQQSYSTPPTFGGGTKVDIK

SEQ ID NO: 45 - protein, 111 aa, synthetic construct
QSALTQPASVSGSPGQSVTISCTGTSSDVGTYNYVSWYQQHPGKAPKLMIYEVSNRPSGVSNRFSGSKSGNTASLTISGLQAEDEADYYCSSYIAYNILEVFGGGTKLTVL

SEQ ID NO: 46 - protein, 106 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNGRTFGQGTKVEIK

SEQ ID NO: 47 - protein, 108 aa, synthetic construct
EIVLTQSPGTLSVSPGERATLSCKASQSISSSSLAWYRQTPGQTPTLLIYGTSTRATGIPDRFSGRGSGTDFTLTITNVKPEDFAVYYCQQYGSSPFTFGPGTRLDFR

SEQ ID NO: 48 - protein, 109 aa, synthetic construct
EVVLTQSPGTLSLSPGERATLSCRASQSVNSIYLAWYQQKPGQAPRLLIYGASNRAAGIPDRFSGSGSGTDFTLTIDRLEPEDFAVYYCQQYDTSPAVTFGPGTKVEIK

SEQ ID NO: 49 - protein, 107 aa, synthetic construct
DIQMTQSPSTLSASVGDRTTITCRASQNIDKWLAWYQQKPGKAPTVLIFKASTLESGVPSRFSGSSSGTEFTLTIDNLQPEDSATYFCHQYSRYPWTFGQGTKVELK

SEQ ID NO: 50 - protein, 108 aa, synthetic construct
QIVLTQSPGTLSLSPGDTATLSCRASQRVTHTHLAWYQQKPGQPPRLLIYATSQRATGIPDRFRGSGSGRDFTLTIASLEPEDFAVYYCQQNGESPRMFGQGTKLDVK

SEQ ID NO: 51 - protein, 110 aa, synthetic construct
QSALSQPASVSGSLGQSITISCSGVGSDAVTYTVVSWYQQHPTKAPKIIISDGDKRPSGVSSRFSGSKSGNTAFLRISGLQTDDEALYYCCSYVGSNTWVFGGGTTVTVL

SEQ ID NO: 52 - protein, 107 aa, synthetic construct
DIQMTQSPSTLSASVGDTVTITCRASQNIHSWLAWYQQKPGQAPKLLIYQASTLQSGVPSRFRGSGSGTEFTLTISSLQPDDFATYHCQQYNFHSPTFGQGTKVEIK

SEQ ID NO: 53 - protein, 113 aa, synthetic construct
AIELTQSPESLAVSPGERATINCKSSQNILYSSNNKNYLAWYQQKVGQPPELLIYWASTRKSGVPDRFSGGGSGTDFTLTISSLQADDVALYYCQQYFYTPWTFGQGTKVEIK

SEQ ID NO: 54 - protein, 111 aa, synthetic construct
QSVLTQPPSVSGGPGQRVTISCTGSTSNIGAGYDVHWYQQLPGTAPKLLIYGNNNRPSGVPDRFSGSKSGTSASLAITGLQAVDEADYYCQSYDSNLSGVVFGGGTKLTVL

SEQ ID NO: 55 - protein, 8 aa, synthetic construct
GFTFNTYE

SEQ ID NO: 56 - protein, 8 aa, synthetic construct
GFTFNTYE

SEQ ID NO: 57 - protein, 8 aa, synthetic construct
GFDFRTYG

SEQ ID NO: 58 - protein, 8 aa, synthetic construct
GFDFRTYS

SEQ ID NO: 59 - protein, 8 aa, synthetic construct
GFNFISYG

SEQ ID NO: 60 - protein, 8 aa, synthetic construct
GGSFSDFY

SEQ ID NO: 61 - protein, 10 aa, synthetic construct
GDPNTISTHN

SEQ ID NO: 62 - protein, 8 aa, synthetic construct
GGTFHTYT

SEQ ID NO: 63 - protein, 8 aa, synthetic construct
GFTFSTNA

SEQ ID NO: 64 - protein, 8 aa, synthetic construct
GFTFSSYE

SEQ ID NO: 65 - protein, 8 aa, synthetic construct
GGSFDTYA

SEQ ID NO: 66 - protein, 10 aa, synthetic construct
GGSIFSSNYY

SEQ ID NO: 67 - protein, 8 aa, synthetic construct
GGTFSTYA

SEQ ID NO: 68 - protein, 8 aa, synthetic construct
GGTFDNSA

SEQ ID NO: 69 - protein, 8 aa, synthetic construct
GTTFTRPY

SEQ ID NO: 70 - protein, 10 aa, synthetic construct
GFSLTTTGVG

SEQ ID NO: 71 - protein, 8 aa, synthetic construct
GGSMNNYY

SEQ ID NO: 72 - protein, 8 aa, synthetic construct
GFTFSSYA

SEQ ID NO: 73 - protein, 6 aa, synthetic construct
QTISNN

SEQ ID NO: 74 - protein, 6 aa, synthetic construct
QSIGNN

SEQ ID NO: 75 - protein, 9 aa, synthetic construct
TGAVTSDHY

SEQ ID NO: 76 - protein, 6 aa, synthetic construct
NIGNKN

SEQ ID NO: 77 - protein, 6 aa, synthetic construct
NIGSKN

SEQ ID NO: 78 - protein, 6 aa, synthetic construct
QGIRNF

SEQ ID NO: 79 - protein, 6 aa, synthetic construct
VMPPRS

SEQ ID NO: 80 - protein, 6 aa, synthetic construct
QSISTH

SEQ ID NO: 81 - protein, 9 aa, synthetic construct
SSDVGTYNY

SEQ ID NO: 82 - protein, 6 aa, synthetic construct
QSVSSN

SEQ ID NO: 83 - protein, 7 aa, synthetic construct
QSISSSS

SEQ ID NO: 84 - protein, 7 aa, synthetic construct
QSVNSIY

SEQ ID NO: 85 - protein, 6 aa, synthetic construct
QNIDKW

SEQ ID NO: 86 - protein, 7 aa, synthetic construct
QRVTHTH

SEQ ID NO: 87 - protein, 9 aa, synthetic construct
GSDAVTYTV

SEQ ID NO: 88 - protein, 6 aa, synthetic construct
QNIHSW

SEQ ID NO: 89 - protein, 12 aa, synthetic construct
QNILYSSNNKNY

SEQ ID NO: 90 - protein, 9 aa, synthetic construct
TSNIGAGYD

SEQ ID NO: 91 - protein, 8 aa, synthetic construct
ISSTGNNI

SEQ ID NO: 92 - protein, 8 aa, synthetic construct
ISGTGNNI

SEQ ID NO: 93 - protein, 8 aa, synthetic construct
ISSTSSAI

SEQ ID NO: 94 - protein, 8 aa, synthetic construct
ISSTSATI

SEQ ID NO: 95 - protein, 8 aa, synthetic construct
ISSASNTI

SEQ ID NO: 96 - protein, 7 aa, synthetic construct
INHSGSS

SEQ ID NO: 97 - protein, 7 aa, synthetic construct
IYYIQEI

SEQ ID NO: 98 - protein, 8 aa, synthetic construct
IIPVFATE

SEQ ID NO: 99 - protein, 8 aa, synthetic construct
ISGSRDRT

SEQ ID NO: 100 - protein, 8 aa, synthetic construct
ISSSGSTI

SEQ ID NO: 101 - protein, 8 aa, synthetic construct
ITPMFGTS

SEQ ID NO: 102 - protein, 7 aa, synthetic construct
VYFSGTT

SEQ ID NO: 103 - protein, 8 aa, synthetic construct
ITPISGAT

SEQ ID NO: 104 - protein, 8 aa, synthetic construct
IIPFVGAA

SEQ ID NO: 105 - protein, 8 aa, synthetic construct
INPTGVNV

SEQ ID NO: 106 - protein, 7 aa, synthetic construct
IYWDDRA

SEQ ID NO: 107 - protein, 7 aa, synthetic construct
ISSGGTT

SEQ ID NO: 108 - protein, 8 aa, synthetic construct
IWFDGSSK

SEQ ID NO: 109-126 - skipped (18 entries)

SEQ ID NO: 127 - protein, 16 aa, synthetic construct
ARGGQLWFGERLNFQH

SEQ ID NO: 128 - protein, 16 aa, synthetic construct
ARGGQLWFGERLYFHF

SEQ ID NO: 129 - protein, 17 aa, synthetic construct
ARGGSLWFGREFHAMDV

SEQ ID NO: 130 - protein, 19 aa, synthetic construct
ARERITVFFRGRSQHASNV

SEQ ID NO: 131 - protein, 19 aa, synthetic construct
ARDIVTIFFRGRSQHAFSV

SEQ ID NO: 132 - protein, 12 aa, synthetic construct
ARKVWPFNGMDV

SEQ ID NO: 133 - protein, 23 aa, synthetic construct
ASEPVKIFGVVSLGEVFYVGMDV

SEQ ID NO: 134 - protein, 18 aa, synthetic construct
ARGRHSVVEPAAKYRFDT

SEQ ID NO: 135 - protein, 12 aa, synthetic construct
ARDIGLGRGHDY

SEQ ID NO: 136 - protein, 18 aa, synthetic construct
ARDFYYDSSGYYPDAFDI

SEQ ID NO: 137 - protein, 20 aa, synthetic construct
AKDWSFDDDGGAYVPSAMDV

SEQ ID NO: 138 - protein, 18 aa, synthetic construct
ARDVRMRSGRPRQYCMDV

SEQ ID NO: 139 - protein, 20 aa, synthetic construct
ARVGQPKFFERSSDGEHFQH

SEQ ID NO: 140 - protein, 16 aa, synthetic construct
ARDGLRLNFYYYGMDV

SEQ ID NO: 141 - protein, 15 aa, synthetic construct
ATSSGVVSLRSAFDI

SEQ ID NO: 142 - protein, 16 aa, synthetic construct
AYRLEHPSNWYDYFHH

SEQ ID NO: 143 - protein, 17 aa, synthetic construct
ARMFYESSTGYPSWFDP

SEQ ID NO: 144 - protein, 17 aa, synthetic construct
ARDPAAGRSYYYYYVDV

SEQ ID NO: 145 - protein, 9 aa, synthetic construct
QQSHSTPLT

SEQ ID NO: 146 - protein, 9 aa, synthetic construct
QQSFSTPIT

SEQ ID NO: 147 - protein, 10 aa, synthetic construct
LLSYSGARRV

SEQ ID NO: 148 - protein, 11 aa, synthetic construct
QVWDSSSDHQV

SEQ ID NO: 149 - protein, 11 aa, synthetic construct
QVWDISSDHQV

SEQ ID NO: 150 - protein, 8 aa, synthetic construct
QQLTNYAT

SEQ ID NO: 151 - protein, 11 aa, synthetic construct
QSTDMNSIYKT

SEQ ID NO: 152 - protein, 9 aa, synthetic construct
QQSYSTPPT

SEQ ID NO: 153 - protein, 11 aa, synthetic construct
SSYIAYNILEV

SEQ ID NO: 154 - protein, 8 aa, synthetic construct
QQYNNGRT

SEQ ID NO: 155 - protein, 9 aa, synthetic construct
QQYGSSPFT

SEQ ID NO: 156 - protein, 10 aa, synthetic construct
QQYDTSPAVT

SEQ ID NO: 157 - protein, 9 aa, synthetic construct
HQYSRYPWT

SEQ ID NO: 158 - protein, 9 aa, synthetic construct
QQNGESPRM

SEQ ID NO: 159 - protein, 10 aa, synthetic construct
CSYVGSNTWV

SEQ ID NO: 160 - protein, 9 aa, synthetic construct
QQYNFHSPT

SEQ ID NO: 161 - protein, 9 aa, synthetic construct
QQYFYTPWT

SEQ ID NO: 162 - protein, 11 aa, synthetic construct
QSYDSNLSGVV

SEQ ID NO: 163 - protein, 19 aa, synthetic construct
CARGGSLWFGREFHAMDVW

SEQ ID NO: 164 - protein, 18 aa, synthetic construct
CARGGQLWFGERLYFHFW

SEQ ID NO: 165 - protein, 18 aa, synthetic construct
CARGGQLWFGERLNFQHW

SEQ ID NO: 166 - protein, 21 aa, synthetic construct
CARDIVTIFFRGRSQHAFSVW

SEQ ID NO: 167 - protein, 21 aa, synthetic construct
CARERITVFFRGRSQHASNVW

SEQ ID NO: 168 - protein, 14 aa, synthetic construct
CARKVWPFNGMDVW

SEQ ID NO: 169 - protein, 25 aa, synthetic construct
CASEPVKIFGVVSLGEVFYVGMDVW

SEQ ID NO: 170 - protein, 12 aa, synthetic construct [sequence omitted]

SEQ ID NO: 171 - protein, 11 aa, synthetic construct [sequence omitted]

SEQ ID NO: 172 - protein, 11 aa, synthetic construct [sequence omitted]

SEQ ID NO: 173 - protein, 13 aa, synthetic construct
CQVWDISSDHQVF

SEQ ID NO: 174 - protein, 13 aa, synthetic construct [sequence omitted]

SEQ ID NO: 175 - protein, 10 aa, synthetic construct
CQQLTNYATF

SEQ ID NO: 176 - protein, 11 aa, synthetic construct
QSTDMNSIYKT

SEQ ID NO: 177 - protein, 19 aa, synthetic construct
CARDPAAGRSYYYYYVDVW

SEQ ID NO: 178 - protein, 19 aa, synthetic construct
CARMFYESSTGYPSWFDPW

SEQ ID NO: 179 - protein, 18 aa, synthetic construct
CAYRLEHPSNWYDYFHHW

SEQ ID NO: 180 - protein, 17 aa, synthetic construct
CATSSGVVSLRSAFDIW

SEQ ID NO: 181 - protein, 18 aa, synthetic construct
CARDGLRLNFYYYGMDVW

SEQ ID NO: 182 - protein, 22 aa, synthetic construct
CARVGQPKFFERSSDGEHFQHW

SEQ ID NO: 183 - protein, 20 aa, synthetic construct
CARDVRMRSGRPRQYCMDVW

SEQ ID NO: 184 - protein, 22 aa, synthetic construct
CAKDWSFDDDGGAYVPSAMDVW

SEQ ID NO: 185 - protein, 20 aa, synthetic construct
CARDFYYDSSGYYPDAFDIW

SEQ ID NO: 186 - protein, 14 aa, synthetic construct
CARDIGLGRGHDYW

SEQ ID NO: 187 - protein, 20 aa, synthetic construct
CARGRHSVVEPAAKYRFDTW

SEQ ID NO: 188 - protein, 13 aa, synthetic construct [sequence omitted]

SEQ ID NO: 189 - protein, 11 aa, synthetic construct
CQQYFYTPWTF

SEQ ID NO: 190 - protein, 11 aa, synthetic construct
CQQYNFHSPTF

SEQ ID NO: 191 - protein, 12 aa, synthetic construct [sequence omitted]

SEQ ID NO: 192 - protein, 11 aa, synthetic construct
CQQNGESPRMF

SEQ ID NO: 193 - protein, 11 aa, synthetic construct
CHQYSRYPWTF

SEQ ID NO: 194 - protein, 12 aa, synthetic construct
CQQYDTSPAVTF

SEQ ID NO: 195 - protein, 11 aa, synthetic construct [sequence omitted]

SEQ ID NO: 196 - protein, 10 aa, synthetic construct [sequence omitted]

SEQ ID NO: 197 - protein, 13 aa, synthetic construct
CSSYIAYNILEVF

SEQ ID NO: 198 - protein, 11 aa, synthetic construct
CQQSYSTPPTF

SEQ ID NO: 199 - DNA, 15 bp, synthetic construct
  source 1..15: mol_type = other DNA
attcgccttacgcaa

SEQ ID NO: 200 - DNA, 15 bp, synthetic construct
  source 1..15: mol_type = other DNA
aacccaccgttgtta

SEQ ID NO: 201 - DNA, 15 bp, synthetic construct
  source 1..15: mol_type = other DNA
ggtagccctagagta

SEQ ID NO: 202 - DNA, 15 bp, synthetic construct
  source 1..15: mol_type = other DNA
cagtaagttcgggac

SEQ ID NO: 203 - DNA, 15 bp, synthetic construct
  source 1..15: mol_type = other DNA
cttcactctgtcagg

SEQ ID NO: 204 - DNA, 15 bp, synthetic construct
  source 1..15: mol_type = other DNA
tacgcctataacttg

SEQ ID NO: 205 - DNA, 15 bp, synthetic construct
  source 1..15: mol_type = other DNA
agactaatagctgac

SEQ ID NO: 206 - DNA, 15 bp, synthetic construct
  source 1..15: mol_type = other DNA
gctcctttacacgta

SEQ ID NO: 207 - DNA, 15 bp, synthetic construct
  source 1..15: mol_type = other DNA
gcagcgtataagtca

SEQ ID NO: 208 - DNA, 15 bp, synthetic construct
  source 1..15: mol_type = other DNA
gctcctttacacgta

SEQ ID NO: 209 - DNA, 15 bp, synthetic construct
  source 1..15: mol_type = other DNA
cttcactctgtcagg

SEQ ID NO: 210 - DNA, 15 bp, synthetic construct
  source 1..15: mol_type = other DNA
tggtaacgacagtcc

SEQ ID NO: 211 - DNA, 15 bp, synthetic construct
  source 1..15: mol_type = other DNA
tgtgtattcccttgt

SEQ ID NO: 212 - DNA, 15 bp, synthetic construct
  source 1..15: mol_type = other DNA
tacgcctataacttg

SEQ ID NO: 213 - DNA, 15 bp, synthetic construct
  source 1..15: mol_type = other DNA
gtgtgttgtcctatg

SEQ ID NO: 214 - DNA, 15 bp, synthetic construct
  source 1..15: mol_type = other DNA
aacccaccgttgtta

SEQ ID NO: 215 - DNA, 15 bp, synthetic construct
  source 1..15: mol_type = other DNA
cagtaagttcgggac

SEQ ID NO: 216 - DNA, 15 bp, synthetic construct
  source 1..15: mol_type = other DNA
gtaagacgcctatgc

SEQ ID NO: 217 - DNA, 15 bp, synthetic construct
  source 1..15: mol_type = other DNA
tttcaacgccctttc

SEQ ID NO: 218 - DNA, 15 bp, synthetic construct
  source 1..15: mol_type = other DNA
ccgtcctgatagatg

SEQ ID NO: 219 - DNA, 15 bp, synthetic construct
  source 1..15: mol_type = other DNA
tcatttcctccgatt

SEQ ID NO: 220 - DNA, 15 bp, synthetic construct
  source 1..15: mol_type = other DNA
ggtagccctagagta

SEQ ID NO: 221 - DNA, 15 bp, synthetic construct
  source 1..15: mol_type = other DNA
agactaatagctgac

SEQ ID NO: 222 - DNA, 15 bp, synthetic construct
  source 1..15: mol_type = other DNA
cagcccactgcaata

SEQ ID NO: 223 - DNA, 15 bp, synthetic construct
  source 1..15: mol_type = other DNA
atcgtcgagagctag

SEQ ID NO: 224 - DNA, 15 bp, synthetic construct
  source 1..15: mol_type = other DNA
tcacagttccttgga

SEQ ID NO: 225 - DNA, 15 bp, synthetic construct
  source 1..15: mol_type = other DNA
cagatgatccaccat

SEQ ID NO: 226 - DNA, 15 bp, synthetic construct
  source 1..15: mol_type = other DNA
gacctcattgtgaat

SEQ ID NO: 227 - DNA, 15 bp, synthetic construct
  source 1..15: mol_type = other DNA
tgaccttcctctcct

SEQ ID NO: 228 - DNA, 15 bp, synthetic construct
  source 1..15: mol_type = other DNA
caggtcccttatttc

SEQ ID NO: 229 - DNA, 15 bp, synthetic construct
  source 1..15: mol_type = other DNA
acaatttgtctgcga

SEQ ID NO: 230 - DNA, 15 bp, synthetic construct
  source 1..15: mol_type = other DNA
aaccttccgtctaag

SEQ ID NO: 231 - DNA, 15 bp, synthetic construct
  source 1..15: mol_type = other DNA
aatcacggtccttgt

SEQ ID NO: 232 - DNA, 47 bp, synthetic construct
  source 1..47: mol_type = other DNA
ccttggcacccgagaattccannnnnnnnnnnnncccatataagaaa